(19) United States
(12) Patent Application Publication
(10) Pub. No.: US 2026/0240884 A1
(43) Pub. Date: Aug. 20, 2026

(54) MIXTURE OF HUMAN MILK OLIGOSACCHARIDES

(71) Applicant: N.V. Nutricia, Zoetermeer (NL)

(72) Inventors: Marko Mank, Utrecht (NL); Atanaska Ivanova Kostadinova, Utrecht (NL); Lauriane Emmanuelle Mélanie Schwebel, Utrecht (NL); Veronica Ayechu Muruzabal, Utrecht (NL); Cornelus Johannes Petrus van Limpt, Utrecht (NL); Bernd Stahl, Utrecht (NL); Ingrid Brunhilde Renes, Utrecht (NL); Nienke Kettelarij, Utrecht (NL); John Mark Anthony Gonsalves, Utrecht (NL); Sibel Göraler, Utrecht (NL); Mirjam Kool, Utrecht (NL)

(73) Assignee: N.V. Nutricia, Zoetermeer (NL)

(21) Appl. No.: 19/646,725

(22) Filed: Apr. 14, 2026

Related U.S. Application Data
(63) Continuation of application No. PCT/EP2024/079733, filed on Oct. 21, 2024.

(30) Foreign Application Priority Data
Oct. 19, 2023 (EP) 23204649.0

(51) Int. Cl. A61K 31/702; A23C 9/20; A23L 33/00; C07H 3/06; C07H 5/04

(52) U.S. Cl. CPC A61K 31/702; A23C 9/206; A23L 33/40; C07H 3/06; C07H 5/04

(57) ABSTRACT

The invention pertains to a synthetic mixture of human milk oligosaccharides (HMOs) wherein the mixture comprises 10-30 wt % acidic HMOs and 70-90 wt % neutral HMOs, wherein the neutral HMOs comprises 75-95 wt % of fucosylated HMOs based on weight of the neutral HMOs and wherein the mixture comprises at least 19 HMOs.

### FIELD OF THE INVENTION

The present invention relates to synthetic mixtures of human milk oligosaccharides and uses thereof in the prevention and treatment of pathogenic infections.

### BACKGROUND OF THE INVENTION

Human milk contains substantial amounts of non-digestible carbohydrates, known as human milk oligosaccharides (HMOs). Mature human milk contains about 5 to 15 g/l of HMOs. It is presumed that more than 200 structurally distinct oligosaccharides are present in human milk. The building blocks of human milk oligosaccharides are the monosaccharides D-glucose (Glc), D-galactose (Gal), N-acetylglucosamine (GlcNAc), L-fucose (Fuc), and sialic acid (N-acetyl neuraminic acid (Neu5Ac). Lactose (Galβ1-4Glc) forms the reducing end and can be elongated with N-acetyllactosamine repeat units (Galβ1-3/4GlcNAc). Lactose or the polylactosamine backbone can be sialylated in α2-3 and/or α2-6 linkages and/or fucosylated in α1-2, α1-3, and/or α1-4 linkages. The structural complexity and abundance of these non-digestible oligosaccharides is unique for human milk as in milk of other mammalian species the level of non-digestible oligosaccharides is much lower. HMOS are known to exert various functions, i.e., anti-infective (against bacteria, viruses, fungi, and parasites), signaling, anti-inflammatory/immunomodulatory, and prebiotic effects, with different HMOs having different specific effects.

Mother's milk is recommended for all infants. However, there are circumstances that make breastfeeding inadequate or less desirable. In those cases, infant formulas are a good alternative. The composition of modern infant formulas is adapted in such a way that it meets many of the special nutritional requirements of the fast growing and developing infant. In the past, infant formulas did not contain non-digestible oligosaccharides. Subsequently infant formulas were developed, containing prebiotic, non-digestible oligosaccharides to functionally mimic the role of the HMOs. One of the best studied mixtures of such prebiotics is a mixture of galacto-oligosaccharides (GOS) plus long chain fructo-oligosaccharides (IcFOS) in a weight ratio of 9:1. Upon administration of this specific prebiotic mixture to infants, bifidobacteria increase and pathogens decrease in the intestinal microbiota, rendering the microbiota more similar to the microbiota of human milk fed infants (Knol et al, Acta Pmdiatrica, 2005; 94 (Suppl 449); Knol et al, 31-33. Pediatr Gastroenterol Nutr, Vol. 40, No. 1, January 2005; WO 2005/039319).

More recently milk oligosaccharides with a structure identical to HMOs have become available as produced by fermentation by genetically modified micro-organisms and infant formulas containing a HMO or a mixture of HMOs have become available. HMOs have been described in the art to be able to reduce the risk of infections by viral and bacterial pathogens. The virulence of enteric viruses and bacteria is dependent, in part, on the pathogen's ability to adhere to epithelial surfaces. There are two proposed mechanisms for how HMOs modulate bacterial and viral pathogenesis. As HMOs share structural homology with epithelial cell surface glycans, they serve as soluble decoy receptors to prevent early cellular attachment. Additionally, HMOs bind epithelial cell surface receptors to block bacterial or viral adhesions. HMOs have been explored to protect against bacterial and virus invasions by mimicking epithelial cell surface glycans.

Additionally, various HMOs possess an anti-inflammatory effect and act as immunomodulators. In line therewith it was proposed that HMOs reduce the risks of developing food allergies. A positive effect of sialylated HMOs on the development of a neonate's central nervous system is also intensely discussed (reviewed in “Prebiotics and Probiotics in Human Milk, Origins and functions of milk-borne oligosaccharides and bacteria”, Academic Press (2017) editors: McGuire M., McGuire M., and Bode L).

Mother's milk is recommended for all infants. However, in some cases breast feeding is inadequate or unsuccessful for medical reasons or the mother chooses not to breast feed. Infant formulae have been developed for these situations.

Milk oligosaccharides with a structure identical to HMOs and infant formulas containing a HMO or a mixture of HMOs have in recent times become available since HMOs can now be produced amongst others by fermentation by genetically modified micro-organisms. To take advantage of the beneficial effects of HMOs, efforts are made in adding individual HMOs to nutritional compositions, in particular to infant formula.

Several compositions have been developed using mixtures of HMOs for different purposes.

WO2004002495 describes an oligosaccharide-containing substance or receptor binding to diarrheagenic Escherichia coli and/or zoonotic Helicobacter species, and use thereof in, e.g., pharmaceutical, nutritional, and other compositions for prophylaxis and treatment of diarrhea, hemorrhagic colitis, or hemolytic uremic syndrome. WO9956754 relates to compositions containing at least one fucose residue in an alpha 1-2 linkage such as 2FL and uses thereof. In particular, such compositions can be used in the treatment and prevention of gastrointestinal infections like diarrhea and enterocolitis. US2014248415 describes several examples of HMOs mixtures, some including both 2FL and LNnT in various ratios. They may be used for various health benefits such as immune system maturation, allergy, influenza, diarrhea.

WO9843495 relates to a process for inhibiting Bacteroides, Clostridium and E. coli infection in a subject comprising feeding the subject a synthetic nutritional formulation that contains an effective anti-bacterial amount of Lacto-N-neoTetraose. By inhibiting the growth of these bacteria, infants are provided with resistance against gastroenteritis.

CN113907144 A describes a combination of 5 HMOs of 53% 2′-FL, 21% 3-FL, 16% LNT, 5% 3′-SL and 5% 6′-SL provides a beneficial effect on Staphylococcus aureus. US2014/248415 A1 describes mixtures containing 4 HMOs with 10% acidic and 90% neutral, of which 91% are fucosylated.

WO2009/077352 relates to a composition suitable in the prevention of pathogenic infections comprising a particular synergistic association of a probiotic Bifidobacterium with a fucosylated oligosaccharide.

WO2014187464 describes synthetic HMO mixtures for treating the microbiota of a human, in particular adults wherein the mixture comprises at least 6 and up to 28 oligosaccharides. Rasmussen et al. J Nutr Biochem 2017 describes the effect of a mixture of 24 HMOs on intestinal function and inflammation after preterm birth in pigs.

There is a need to develop mixtures of HMOs that will be particularly efficient and adapted to prevent or treat a broad range of infections/inflammations.

There is a continuing need to establish nutritional solutions that provide combinations of different HMOs to more closely resemble the natural source of HMOs, i.e., human milk, and that help to protect against infections in a better way than compositions containing only a single or a mixture of a limited number of species of HMOs.

### SUMMARY OF THE INVENTION

The present invention concerns synthetic mixtures of human milk oligosaccharides (HMOs) and nutritional compositions comprising said synthetic mixtures of HMOs. More particularly the invention relates to a novel synthetic mixture of HMOs and nutritional compositions comprising said synthetic mixture of HMOs exerting a broad preventive and treating effect against infections, preferably pathogenic infections.

The present inventors have found that a synthetic mixture of HMOs comprising 10-30 wt % acidic HMOs and 70-90 wt % neutral HMOs provided beneficial preventive and treatment effects against pathogenic infections, preferably against viral, bacterial, fungal and/or parasitic infections, more preferably at least viral and bacterial infections. It was observed by the inventors that such a synthetic mixture of HMOs provided an improved protection both in terms of prevention and treatment against infections than oligosaccharide mixtures of less than 6 different oligosaccharides and that an effect closer to the full spectrum of HMOs extracted from breast milk is obtained.

The synthetic mixture of HMOs according to the invention comprises 10-30 wt % acidic HMOs and 70-90 wt % neutral HMOs. In a preferred aspect, the synthetic mixture of HMOs comprises 19 HMOs.

In a preferred aspect the mixture of HMOs comprises 10-30 wt % acidic HMOs and 70-90 wt % neutral HMOs wherein the neutral HMOs comprises 75-95 wt % of fucosylated HMOs based on weight of the neutral HMOs. In a further aspect the invention pertains to the mixture of HMOs comprising 10-30 wt % acidic HMOs and 70-90 wt % neutral HMOs, wherein the acidic HMOs comprise at least 3′-sialyllactose (3′-SL) and 6′-sialyllactose (6′-SL) and wherein the weight ratio of 3′SL to 6′SL is between 0.5:3 to 1:1, preferably between 1:3 to 1:2, even more preferably about 1:2.

In a further preferred aspect the invention pertains to said mixture of HMOs comprising 10-30 wt % acidic HMOs and 70-90 wt % neutral HMOs, wherein the acidic HMOs are selected from 3′sialyllactose (3′SL), 6′-sialyllactose (6′SL), sialyllacto-N-tetraose a (LSTa), sialyllacto-N-tetraose b (LSTb), sialyllacto-N-tetraose c (LSTc) and disialyllacto-N-tetraose (DSLNT), and wherein the neutral HMOs are selected from 2′-fucosyllactose (2′FL), 3′-fucosyllactose (3′FL), difucosyllactose (DFL), lacto-N-tetraose (LNT), lacto-N-neotetraose (LNnT), lacto-N-fucosylpentaose-I (LNFP I), lacto-N-fucosylpentaose-II (LNFP II), lacto-N-fucosylpentaose III (LNFP III), lacto-N-fucosylpentaose V (LNFP V), lacto-N-difucosylhexaose I (LNDFH I, lacto-N-neodifucosylhexaose-I (LNnDFH I) and lacto-N-difucosylhexaose-II (LNDFH II) and Lacto-N-neodifucohexaose II (LNnDFH II).

In a preferred aspectthe mixture of HMOs comprises, preferably consists essentially of 3′SL, 6′SL, LSTa, LSTb, LSTc, DSLNT, 2′FL, 3′FL, DFL, LNT, LNnT, LNFP I, LNFP II, LNFP III, LNFP V, LNDFH I, LNnDFH I and LNDFH II/LNnDFH II.

### LIST OF FIGURES

The present invention will be discussed in more detail below, with reference to the attached figures.

FIG. 1 shows the results of pre-incubation of SARS-Cov2 WA1 with different concentrations of 2′-FL, GOS-FOS in a 9:1 ratio, total HMOs, a mixture of 19 HMOs or 6′-SL on viral RNA as assessed by RT-qPCR. The results are shown against the percentage non-treated control and normalized to the lowest concentration.

FIG. 2 shows A) measurements of the transepithelial electrical resistance (TEER) and B) lucifer yellow (LY) permeability upon exposure of Caco-2 cells to a mixture of 19 HMO, a mixture of 5 HMO, medium only, lactose, cellobiose or lactoferrin followed by infection with SA11 rotavirus.

### LIST OF PREFERRED EMBODIMENTS

1. A synthetic mixture of human milk oligosaccharides (HMOs) comprising 10-30 wt % acidic HMOs and 70-90 wt % neutral HMOs, wherein the neutral HMOs comprises 75-95 wt % of fucosylated HMOs based on weight of the neutral HMOs, for use in the treatment and/or prevention of infections, preferably pathogenic infections, wherein the mixture preferably comprises 19 HMOs.
2. The synthetic mixture of HMOs for use according to embodiment 1, wherein the acidic HMOs comprise at least 3′-sialyllactose (3′-SL) and 6′-sialyllactose (6′-SL) and wherein the weight ratio of 3′SL to 6′SL is between 0.5:3 to 1:1, preferably between 1:3 to 1:2, even more preferably about 1:2.
3. The synthetic mixture of HMOs for use according to embodiments 1 and 2, wherein the use in the treatment and/or prevention of infections, preferably pathogenic infections.
4. The synthetic mixture of HMOs for use according to the preceding embodiments, wherein the pathogenic infections are selected from viral and/or bacterial infections, preferably pathogenic infections with a virus selected from double-stranded RNA viruses, single-stranded positive and negative sense RNA viruses, and DNA viruses and/or bacteria selected from gram-positive and gram-negative bacteria.
5. The synthetic mixture of HMOs for use according to any one of the preceding embodiments, wherein the use in the treatment and/or prevention of infections, preferably pathogenic infections is against a virus selected from rotavirus, respiratory syncytial virus (RSV) and SARS-COV2 and/or bacterium selected from L. monocytogenes, E. coli, S. aureus, S. typhimurium, L. monocytogenes, E. faecium, E. faecalis, P. mirabilis and K. pneumoniae, preferably E. coli.
6. The synthetic mixture of HMOs for use according to any one of the preceding embodiments wherein the treatment and/or prevention of infections, preferably pathogenic infections is more similar to the treatment and/or prevention of infections observed in human milk fed infants and/or improved compared to the treatment and/or prevention of infections in infants administered a composition not comprising the mixture of HMOs.
7. The synthetic mixture of HMOs for use according to any one of the preceding embodiments, wherein the neutral HMOs comprise at least LNT and LNnT and wherein the weight ratio of LNT to LNnT is between 12:1 and 1:1, preferably between 10:1 and 3:1, even more preferably between 9:1 to 6:1.
8. The synthetic mixture of HMOs for use according to any one of the preceding embodiments, wherein HMOs are selected from 3′SL, 6′SL, LSTa, LSTb, LSTc, DSLNT, 2′FL, 3′FL, DFL, LNT, LNnT, LNFP I, LNFP II, LNFP III, LNFP V, LNDFH I, LNnDFH I, LNDFH II and LNnDFH II.
9. The synthetic mixture of HMOs for use according to any of the preceding embodiments, wherein the mixture consists of 2′-FL, 3′FL, 3′-SL, 6′-SL, DFL, LNT, LnNT, LNFP I, LNFP II, LNFP III, LSTc, LNDFH I and DSLNT.
10. The synthetic mixture of HMOs for use according to any one of the preceding embodiments, wherein the mixture of HMOs is comprised in a nutritional composition, wherein the nutritional composition is selected from infant formula, follow-on formula, and young-child formula.
11. Nutritional composition comprising the synthetic mixture of HMOs for use according to any one of the preceding embodiments, wherein the nutritional composition further comprising group II non-digestible saccharides selected from the group consisting of fructo-oligosaccharides, non-digestible dextrins, galacto-oligosaccharides (such as betagalacto-oligosaccharides), xylo-oligosaccharides, arabino-oligosaccharides, arabinogalacto-oligosaccharides, gluco-oligosaccharides, gentio-oligosaccharides, glucomanno-oligosaccharides, galactomanno-oligosaccharides, mannan-oligosaccharides, isomalto-oligosaccharides, nigero-oligosaccharides, chito-oligosaccharides, soy oligosaccharides, uronic acid oligosaccharides, and mixtures thereof.
12. Nutritional composition comprising the synthetic mixture of HMOs for use according to any one of the preceding embodiments wherein the total weight ratio of the synthetic mixture of HMOs and the group II non-digestible saccharides of short chain oligosaccharides having a DP between 3 and 6 (DP 3-6) to long chain oligosaccharides have a DP of 7 and higher is from 5:1 to 12:1, preferably from 8:1 to 10:1, even more preferably in a ratio of about 9:1.
13. Synthetic mixture of HMOs wherein the mixture comprises 10-30 wt % acidic HMOs and 70 90 wt % neutral HMOs, wherein the neutral HMOs comprises 75-95 wt % of fucosylated HMOs based on weight of the neutral HMOs and wherein the mixture comprises at least 19 HMOs.
14. Synthetic mixture of HMOs comprising or consisting essentially of 3′SL, 6′SL, LSTa, LSTb, LSTc, DSLNT, 2′FL, 3′FL, DFL, LNT, LNnT, LNFP I, LNFP II, LNFP III, LNFP V, LNDFH I, LNnDFH I and LNDFH II and LNnDFH II.
15. Synthetic mixture of HMOs according to embodiment 13 wherein the mixture comprises 10-30 wt % acidic HMOs and 70-90 wt % neutral HMOs, and wherein the neutral HMOs comprises 75-95 wt % of fucosylated HMOs based on weight of the neutral HMOs.
16. Synthetic mixture of HMOs according to any one of embodiments 13 and 15 wherein the weight ratio of 3′SLto 6′SL is between 0.5:3 to 1:1, preferably between 1:3 to 1:2, even more preferably about 1:2.
17. Synthetic mixture of HMOs according to any one of embodiments 13 to 16 wherein the synthetic mixture of HMOs comprises, preferably consists essentially of
(i) 1.4 to 2.6 wt % 3′-SL, more preferably 1.6 to 2.4 wt %, even more preferably 1.8 to 2.2 wt %;
(ii) 3.5 to 4.7 wt % 6′SL, more preferably 3.7 to 4.5 wt %, even more preferably 3.9 to 4.3 wt %;
(iii) 0.8 to 1.6 wt % LSTa, more preferably 0.9 to 1.5 wt %, even more preferably 1.0 to 1.4 wt %;
(iv) 0.7 to 1.4 wt % LSTb, more preferably 0.8 to 1.3 wt %, even more preferably 0.9 to 1.2 wt %;
(v) 4.1 to 4.8 wt % LSTc, more preferably 4.2 to 4.7 wt %, even more preferably 4.3 to 4.6 wt %;
(vi) 7.8 to 11.2 wt % DSLNT, more preferably 8.0 to 11.0 wt %, even more preferably 8.5 to 10.5 wt %;
(vii) 25 to 32 wt % 2′FL, more preferably 26 to 31 wt %, even more preferably 27 to 30 wt %;
(viii) 19 to 25 wt % 3′FL, more preferably 20 to 24 wt %, even more preferably 20.5 to 23 wt %;
(ix) 4.1 to 4.8 wt % DFL, more preferably 4.2 to 4.7 wt %, even more preferably 4.3 to 4.6 wt %;
(x) 5 to 10 wt % LNT, more preferably 6 to 9 wt %, even more preferably 7 to 8 wt %;
(xi) 0.5 to 1.1 wt % LNnT, more preferably 0.6 to 1 wt %, even more preferably 0.7 to 0.9 wt %;
(xii) 2.9 to 3.6 wt % LNFP I, more preferably 3 to 3.5 wt %, even more preferably 3.1 to 3.4 wt %;
(xiii) 3.8 to 4.4 wt % LNFP II, more preferably 3.9 to 4.3 wt %, even more preferably 4 to 4.2 wt %
(xiv) 2.9 to 3.6 wt % LNFP III, more preferably 3 to 3.5 wt %, even more preferably 3.1 to 3.4 wt %;
(xv) 0.2 to 0.5 wt % LNFP V, more preferably 0.25 to 0.45 wt %, even more preferably 0.3 to 0.4 wt %;
(xvi) 4.2 to 5 wt % LNDFH I, more preferably 4.4 to 4.8 wt %, even more preferably 4.5 to 4.75 wt %;
(xvii) 0.00001 to 0.02 wt % LNnDFH I, more preferably 0.0001 to 0.015 wt %, even more preferably 0.001 to 0.01 wt %; and
(xviii) 0.7 to 1.3 wt of the combination of LNDFH II and LNnDFH II, more preferably 0.8 to 1.2 wt %, even more preferably 0.9 to 1.15 wt %;
based on total dry weight of the synthetic HMO mixture.
18. Nutritional composition comprising the synthetic HMO mixture according to any one of embodiments 13 to 17, wherein the nutritional composition is selected from infant formula, follow-on formula, and young child milk.

### DETAILED DESCRIPTION OF THE INVENTION

### Definitions

Throughout this application and as used herein, the following terms have the following meanings. An infant is a child under the age of 12 months.

“Infant formula” or “follow-on formula” or “young child formula” means that it concerns a composition that is artificially made or in other words that it is a synthetic composition (i.e., the synthetic composition is not breast milk). Hence the nutritional composition that is administered is an artificial infant formula or an artificial follow-on formula or an artificial young child formula or a synthetic infant formula or a synthetic follow-on formula or a synthetic young child formula. Infant formula refers to nutritional compositions, artificially made, intended for infants of 0 months to about 4 months to 6 months of age and are intended as a substitute for human milk. Typically, infant formulas are suitable to be used as sole source of nutrition. Such infant formulas are also known as starter formula. Follow-on formulas are for infants starting with at 4 months to 6 months of life to 12 months of life and are intended to be supplementary feedings for infants that start weaning on other foods. Infant formulas and follow-on formulas are subject to strict regulations, for example for the EU regulations no. 609/2013 and no. 2016/127.

A young child is a child aged between one and three years, also called a toddler.

Young child formula refers to nutritional compositions, artificially made, intended for children of 12 months up to 48 months of age, which are intended to be supplementary feedings for infants.

“Nutritional composition” means a substance or formulation that satisfies at least a portion of a subject's nutrient requirements. The nutritional composition according to the present invention is preferably selected from an infant formula, a follow-on formula, and a growing-up milk. This means that the present nutritional composition is not human milk. Alternatively, the term “formula” means that it concerns a composition that is artificially made or in other words that it is synthetic. Hence in one embodiment the nutritional composition is selected from an artificial infant formula, an artificial follow-on formula and an artificial growing-up milk or a synthetic infant formula, a synthetic follow-on formula and a synthetic growing-up milk.

The term HMO or HMOs refer to human milk oligosaccharide(s). HMOs are complex carbohydrates found in human breast milk ((Urashima et al.: Milk Oligosaccharides. Nova Science Publisher (2011); Chen Adv. Carbohydr. Chem. Biochem. 72, 113 (2015)). These carbohydrates are resistant to enzymatic hydrolysis by digestive enzymes. Each oligosaccharide is based on a combination of glucose, galactose, sialic acid (N-acetylneuraminic acid), fucose and/or N-acetylglucosamine.

HMOs can be divided in neutral or non-acidic HMOs which can either be fucosylated or non-fucosylated, and acidic HMOs that have at least one sialyl residue in their structure. As used herein none of the individual HMOs in the synthetic mixture of HMOs correspond to the group II non-digestible oligosaccharides. In the context of the present invention lactose is not regarded as an HMO species.

Examples of neutral non-fucosylated HMOs include lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH).

A “fucosylated oligosaccharide” is an oligosaccharide having a fucose residue. Examples of fucosylated HMOs include 2′-fucosyllactose (2′-FL), lacto-N-fucopentaose I (LNFP-1), lacto-N-difucohexaose I (LNDFH-1), 3-fucosyllactose (3-FL), difucosyllactose (DFL), lacto-N-fucopentaose II (LNFP-II), lacto-N-fucopentaose III (LNFP-III), lacto-N-difucohexaose 11 (LNDFH-III), lacto-N-difucosylhexaose-II (LNDFH 11), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-paralacto-N-neohexaose II (F-pLNnH II) and fucosyl-lacto-N-neohexaose (FLNnH).

A “sialylated oligosaccharide” is a charged sialic acid containing oligosaccharide, i.e., an oligosaccharide having a sialic acid residue. It has an acidic nature. Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-1), sialyl-lacto-N-neohexaose II (SLNH-II) and disialyl-lacto-N-tetraose (DSLNT).

The HMOs as used herein are given their commonly used name that corresponds to the molecular name as provided in Table 1.

TABLE 1
Names of various HMO molecules

| Common HMO name | Alternative name |
|---|---|
| 2′-FL | Fucα1-2Galβ1-4Glc |
| 3′-FL | Galβ1-4(Fucα1-3)Glc |
| 3′-SL (acidic) | Neu5Ac(a2-3)Gal(b1-4)Glc |
| 3′-sialyllactose | |
| 6′-SL (acidic) | Neu5Ac(a2-6)Gal(b1-4)Glc |
| 6′-sialyllactose | |
| DFL | Fucα1-2Galβ1-4(Fucα1-3)Glc |
| difucosyllactose | |
| LNT | Fucα1-2Galβ1-4(Fucα1-3)Glc |
| lacto-N-tetraose | |
| LNnT | Gal(b1-4)GlcNAc(b1-3)Gal(b1-4)Glc |
| lacto-N-neotetraose | |
| LNFP I | Fucα1-2Galβ1-3GlcNAcβ1-3Galβ1-4Glc, |
| Lacto-N-fucosylopentaose I | |
| LNFP II | Galβ1-3(Fucα1-4)GlcNAcβ1-3Galβ1-4Glc |
| Lacto-N-fucosylopentaose II | |
| LNFP III | Galβ1-4(Fucα1-3)GlcNAcβ1-3Galβ1-4Glc |
| Lacto-N-fucosylpentaose III | |
| LNFP V | Galβ1-3GlcNAcβ1-3Galβ1-4(Fucα1-3)Glc |
| Lacto-N-fucosylpentaose V | |
| LSTa (acidic) | Neu5Acα2-3Galβ1-3GlcNAcβ1-3Galβ1-4Glc |
| sialyllacto-N-tetraose a | |
| LSTb (acidic) | Neu5Ac(a2-6)[Gal(b1-3)]GlcNAc(b1-3)Gal(b1-4)Glc |
| sialyllacto-N-tetraose b | |
| LSTc (acidic) | Neu5Ac(a2-6)Gal(b1-4)GlcNAc(b1-3)Gal(b1-4)Glc |
| sialyllacto-N-tetraose c | |
| LNDFH I | Fuc(a1-2)Gal(b1-3)[Fuc(a1-4)]GlcNAc(b1-3)Gal(b1- |
| Lacto-N-difucosylhexaose I (Le b hexa) | 4)Glc |
| LNnDFH I | Galβ1-4(Fucα1-3)GlcNAcβ1-3Galβ1-4(Fucα1-3)Glc |
| Lewis x (LeX) hexaose/Lacto-N- | |
| neodifucosylhexaose | |
| LNDFH II and LNnDFH II | Fucα1-2Galβ1-3(Fucα1-4)GlcNAcβ1-3Galβ1-4Glc |
| Lacto-N-difucosylhexaose II (Le a hexa) and | and Galβ1-3(Fucα1-4)GlcNAcβ1-3Galβ1-4(Fucα1- |
| Lacto-N-neodifucohexaose II (Le x hexa) | 3)Glc |
| DSLNT (acidic) | Neu5Ac(a2-3)Gal(b1-3)[Neu5Ac(a2-6)]GlcNAc(b1- |
| Disialyllacto-N-tetraose | 3)Gal(b1-4)Glc |

As used herein, a “therapeutically effective amount” is an amount that reduces symptoms, manages, increases, or induces detoxification or provides a nutritional, physiological, or medical benefit associated therewith to the individual.

As used herein the term “synthetic HMO mixture” means HMO mixtures that are not chemically identical to mixtures naturally occurring in the specific ratios in mammalian milks and that can be obtained by chemical and/or biological processes and/or by blending of mammalian milk-derived purified or isolated HMOs. The terms “mixture of HMO” and “HMO mixture” are used interchangeably and both mean the synthetic mixture of HMOs.

HMOs may be isolated by chromatographic or filtration technology from a natural source such as animal milk and several may be produced by biotechnological means known in the art. Several HMOs such as but not limited to 2′-FL, 3-FL, 3′-SI, 6′-SL, LNT, LNnT and DFL are commercially available and can be obtained from commercial sources such as Chr Hansen, BASF, DSM-Glycom.

In this document and in its claims, the verb “to comprise” and its conjugations is used in its non-limiting sense to mean that items following the word are included, but items not specifically mentioned are not excluded. All percentages are by weight unless otherwise stated. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The present invention concerns a synthetic mixture of human milk oligosaccharides (HMOs). In a preferred aspect the synthetic mixture of HMOs and nutritional compositions comprising the synthetic mixture of HMOs is for infants or young children. It has been surprisingly discovered that a HMO mixture according to the invention can provide anti-infective composition for preventing or treating pathogenic infections, preferably bacterial and/or viral infections, more preferably at least bacterial or viral infections. It is believed that the HMO mixture allows for a broad protection against a wide spectrum of pathogens through specific modulation of the intestinal microbiota, binding of viruses, improvement of intestinal barrier function. Further, the HMO mixture of this invention acts as a decoy receptor prevent adherence of pathogenic microorganisms to a subject's cells. These properties make the HMO mixture according to the invention suitable for preventing and treating pathogenic infections. It was discovered that the HMO mixture provided an improved protection both in terms of prevention and treatment against pathogenic infections than oligosaccharide mixtures of less than 6 different oligosaccharides such as 2′-FL, a mixture of 5 HMOs or a combination of GOS and FOS and that an effect closer to human breast milk is obtained.

In a preferred aspect said synthetic mixture of HMOs comprises 10-30 wt % acidic HMOs and 70-90 wt % neutral HMOs. In a further aspect the synthetic mixture of HMOs comprises 10-30 wt % acidic HMOs and 70-90 wt % neutral HMOs, wherein the neutral HMOs comprises 75-95 wt % of fucosylated HMOs based on weight of the neutral HMOs. In a preferred aspect the synthetic mixture of HMOs according to the invention comprises 10-30 wt % acidic HMOs and 70-90 wt % neutral HMOs and preferably comprises 19 HMOs. In a preferred aspect, the synthetic mixture of HMOs comprises 19 HMOs.

In a further aspect the invention pertains to the synthetic mixture of HMOs comprising 10-30 wt % acidic HMOs and 70-90 wt % neutral HMOs, wherein the acidic HMOs comprise at least 3′-sialyllactose (3′-SL) and 6′-sialyllactose (6′-SL) and wherein the weight ratio of 3′SL to 6′SL is between 0.5:3 to 1:1, preferably between 1:3 to 1:2, even more preferably about 1:2. An increased presence of 6′-SL as compared to 3′-SL is believed to provided beneficially increased anti-inflammatory properties in the gastrointestinal tract.

In a further preferred aspect the invention pertains to a synthetic mixture of HMOs comprising 10-30 wt % acidic HMOs and 70-90 wt % neutral HMOs, wherein the acidic HMOs are selected from 3′-sialyllactose (3′SL), 6-sialyllactose (6′SL), sialyllacto-N-tetraose a (LSTa), sialyllacto-N-tetraose b (LSTb), sialyllacto-N-tetraose c (LSTc) and disialyllacto-N-tetraose (DSLNT), and wherein the neutral HMOs are selected from 2′-fucosyllactose (2′FL), 3′-fucosyllactose (3′FL), difucosyllactose (DFL), lacto-N-tetraose (LNT), lacto-N-neotetraose (LNnT), lacto-N-fucosylpentaose-I (LNFP I), lacto-N-fucosylpentaose-II (LNFP II), lacto-N-fucosylpentaose III (LNFP III), lacto-N-fucosylpentaose V (LNFP V), lacto-N-difucosylhexaose I (LNDFH I) lacto-N-neodifucosylhexaose-I (LNnDFH I) and lacto-N-difucosylhexaose-II (LNDFH II) and Lacto-N-neodifucosylhexaose II (LNnDFH II). The synthetic mixture of HMOs according to the invention thus comprises 10-30 wt % acidic HMOs and 70-90 wt % neutral HMOs and comprises preferably 19 HMOs.

In a preferred aspect the synthetic mixture of HMOs comprises, preferably consists essentially of 3′SL, 6′SL, LSTa, LSTb, LSTc, DSLNT, 2′FL, 3′FL, DFL, LNT, LNnT, LNFP I, LNFP II, LNFP III, LNFP V, LNDFH I, LNnDFH I and LNDFH II/LNnDFH II.

### Synthetic Mixture of HMOs

Provided herein is thus a synthetic mixture of HMOs preferably comprising 10-30 wt % acidic HMOs and 70-90 wt % neutral HMOs. In a preferred aspect the synthetic mixture of HMOs according to the invention comprises 10-30 wt % acidic HMOs and 70-90 wt % neutral HMOs and comprises preferably 19 HMOs. In a preferred aspect, the synthetic mixture of HMOs comprises 19 HMOs.

“Synthetic HMO mixture” means HMO mixtures that are not chemically identical to mixtures naturally occurring in the specific ratios in mammalian milks and that can be obtained by chemical and/or biological processes and/or by blending of mammalian milk-derived purified or isolated HMOs. The synthetic HMO mixture may thus be a mixture of naturally occuring and purified or isolated HMOs and synthetically prepared HMOs.

Without wishing to be bound by theory, the inventors believe that the efficacy of the synthetic mixture of HMOs in the prevention and treatment of infections, preferably pathogenic infections, may be the result of the ratios of the individual HMOs together with broad variety in HMO structures which provides for a broad spectrum of immune system-associated effects as well as decoy receptors. It was observed by the inventors that such a mixture of HMOs provided an improved protection both in terms of prevention and treatment against infections than oligosaccharide mixtures of less than 6 different oligosaccharides such as 2′-FL, a mixture of 5 HMOs or a combination of GOS and FOS and that an effect closer to human breast milk is obtained. The inventors of the HMO synthetic have found a mixture of HMOs that does not correspond to the composition of human breast milk, in particular some of the ratios of the individual HMOs are not observed as such in human breast milk of a specific lactation stage. Since it is not always apparent which is the cause underlying a pathogenic infection while determination of the exact pathogen is expensive and time consuming the use of the mixture of HMOs with a broad spectrum of action beneficially improves infection prevention and treatment.

The HMOs may be obtained by any suitable method. Suitable methods for synthesizing HMOs will be well known to those of skill in the art. For example, processes have been developed for producing HMOs by microbial fermentations, enzymatic processes, chemical syntheses, or combinations of these technologies (Zeuner et al., 2019. Molecules, 24(11), p.2033).

In a preferred aspect the mixture of HMOs comprises 10-30 wt %, more preferably 15-25 wt %, even more preferably 20-25 wt % acidic HMOs and 70-90 wt %, more preferably 75 to 85 wt %, even more preferably 75-80 wt % neutral HMOs based on total weight of the HMO mixture.

Preferably the neutral HMOs in the HMO mixture comprise 75-95 wt %, more preferably 80-95 wt %, even more preferably 85-90 wt % of fucosylated HMOs based on weight of the neutral HMOs of the HMO mixture.

In a further embodiment the HMO mixture preferably comprises 60-80 wt %, more preferably 65-75 wt %, even more preferably 67-72 wt % fucosylated HMOs based on weight of the HMO mixture.

In a preferred aspect the HMO mixture comprises 2′-FL and 3′FL in a weight ratio between 5:1 and 1:1, preferably between 4:1 and 1:1, even more preferably between 2:1 and 1:1. The provision of 2′-FL and 3′-FL in the claimed weight ratio is believed to provide complementary beneficial effects in interference with the attachment of pathogens and to provide complementary immunomodulatory effects.

In a further preferred embodiment, the HMO mixture comprises Lacto-N-fucopentaose I (LNFP I) and Lacto-N-fucopentaose II (LNFP III), wherein the HMO mixture comprises LNFP I and LNFP II in a weight ratio between 0.5:3 and 1:1.5, preferably between 1:3 and 1:1, even more preferably 1:2 and 1.1. The provision of LNFP I and LNFP II in the claimed ratio's is believed to provide complementary beneficial effects in interference with the attachment of pathogens and to provide complementary immunomodulatory effects. While LNFPI has been shown to inhibit the adhesion of pathogens, such as enteropathogenic Escherichia coli (EPEC) and protozoan parasite E. histolytica to intestinal cells (Bode et al. Advances in Nutrition, vol 3, 2012), LNFP II has been associated with a reduction in the adhesion of certain pathogens, such as Campylobacter jejuni and Salmonella enterica, to intestinal cells.

In some embodiments the neutral HMOs in the HMO mixture further are N-acetylated oligosaccharides that are at least a mixture of LNT and LNnT. In some particular embodiments the HMO mixture comprises LNT and LNnT, wherein the HMO mixture comprises LNT:LNnT in a weight ratio between 12:1 and 1:1, preferably between 10:1 and 3:1, even more preferably between 9:1 and 6:1. The provision of LNFPI and LNFP III in the claimed ratio's is also believed to provide complementary beneficial effects in interference with the attachment of pathogens and to provide complementary immunomodulatory effects. LNT reduces adhesion of amongst others protozoan parasite E. histolytica whereas LNnT reduces Streptococcus pneumoniae load in rabbit lungs.

In a further aspect the invention pertains to the mixture of HMOs comprising 10-30 wt % acidic HMOs and 70-90 wt % neutral HMOs, wherein the acidic HMOs comprise at least 3′-sialyllactose (3′-SL) and 6′-sialyllactose (6′-SL) and wherein the weight ratio of 3′SLto 6′SL is between 0.5:3 and 1:1, preferably between 1:3 and 1:2, even more preferably about 1:2.

In a further preferred embodiment the HMO mixture comprises disialyllacto-N-tetraose (DSLNT) and sialyllacto-N-tetraose c (LSTc) wherein the weight ratio DSLNT to LSTc is between 3:0.5 and 1:1, preferably between 3:1 and 2:1, even more preferably between 2.5:1 and 2:1. The provision of DSLNT and LSTc in the claimed ratio's is believed to provide complementary beneficial effects in providing a potent interference with the attachment of pathogens. While DSLNT is associated with reduced risk of NEC and is a potent decoy ligand for pathogen binding host cells (Bode L, Front. Pediatr., Sec. Neonatology, Vol 6, 2018), LSTc has a protective role against infections with human JC polyomavirus (Neu U et al., Cell Host & Microbe 8(4): 309-319, 2010) In a further aspect the invention pertains to nutritional compositions comprising said mixture of HMOs and wherein the total ratio of short chain (DP 3-6) to long chain oligosaccharides (DP 7 and higher), based on the total amount of oligosaccharides present in said nutritional composition is from 1:99 to 99:1, more preferably from 1:19 to 19:1, more preferably from 1:1 to 19:1, more preferably from 2:1 to 15:1, more preferably from 5:1 to 12:1, even more preferablyfrom 8:1 to 10:1, even more preferably in a ratio of about 9:1. In a preferred aspect the ratio of short chain (DP 3-6) to long chain oligosaccharides is 9:1.

In a preferred embodiment the fucosylated HMOs of the HMO mixture comprise a combination of 2′-FL, LNFPI and LNDFH I. In a further preferred embodiment, the fucosylated HMOs of the HMO mixture comprise a combination of 3′-FL, LNFP III, LNFP V and LNdFH II. It is believed thatthe provision of HMOs having a similar fucosylbond and ranging from DP3 to DP6 provides for a more potent and sustained protection against pathogens over time.

In a preferred aspect the mixture of HMOs comprises at least
1.4 to 2.6 wt % 3′-SL, more preferably 1.6 to 2.4 wt %, even more preferably 1.8 to 2.2 wt %;
3.5 to 4.7 wt % 6′SL, more preferably 3.7 to 4.5 wt %, even more preferably 3.9 to 4.3 wt %.

In a further preferred aspect, the mixture of HMOs comprises at least
1.4 to 2.6 wt % 3′-SL, more preferably 1.6 to 2.4 wt %, even more preferably 1.8 to 2.2 wt %;
3.5 to 4.7 wt % 6′SL, more preferably 3.7 to 4.5 wt %, even more preferably 3.9 to 4.3 wt %, wherein the weight ratio of 3′SL to 6′SL is between 0.5:3 to 1:1, preferably between 1:3 to 1:2, even more preferably about 1:2.

The acidic HMOs are preferably selected from 3′SL, 6′SL, sialyllacto-N-tetraose a (LSTa), sialyllacto-N-tetraose b (LSTb), sialyllacto-N-tetraose c (LSTc) and disialyllacto-N-tetraose (DSLNT), preferably the acidic HMOs of the HMO mixture are at least 3′-SL and 6′-SL, more preferably at least 3′-SL, 6′-SL, DSLNT and LSTc.

The neutral HMOs are preferably selected from 2′-fucosyllactose (2′FL), 3′-fucosyllactose (3′FL), difucosyllactose (DFL), lacto-N-tetraose (LNT), lacto-N-neotetraose (LNnT), lacto-N-fucosylpentaose-I (LNFP I), lacto-N-fucosylpentaose-II (LNFP II), lacto-N-fucosylpentaose III (LNFP III), lacto-N-fucosylpentaose V (LNFP V), lacto-N-difucosylhexaose I (LNDFH I), lacto-N-neodifucosylhexaose-I (LNnDFH I) and lacto-N-difucosylhexaose-II (LNDFH II) and Lacto-N-neodifucohexaose II (LNnDFH II).

In a further preferred aspect the invention pertains to said mixture of HMOs comprising 10-30 wt % acidic HMOs and 70-90 wt % neutral HMOs, wherein the acidic HMOs are selected from 3′SL, 6′SL, sialyllacto-N-tetraose a (LSTa), sialyllacto-N-tetraose b (LSTb), sialyllacto-N-tetraose c (LSTc) and disialyllacto-N-tetraose (DSLNT), and wherein the neutral HMOs are selected from 2′-fucosyllactose (2′FL), 3′-fucosyllactose (3′FL), difucosyllactose (DFL), lacto-N-tetraose (LNT), lacto-N-neotetraose (LNnT), lacto-N-fucosylpentaose-I (LNFP I), lacto-N-fucosylpentaose-II (LNFP II), lacto-N-fucosylpentaose III (LNFP III), lacto-N-fucosylpentaose V (LNFP V), lacto-N-difucosylhexaose I (LNDFH I), lacto-N-difucosylhexaose-I (LNnDFH I) and lacto-N-difucosylhexaose-II (LNDFH II) and Lacto-N-neodifucohexaose II (LNnDFH II).

The mixture of HMOs according to the invention thus comprises 10-30 wt % acidic HMOs and 70-90 wt % neutral HMOs and comprises preferably 19 HMOs.

In a preferred aspect the mixture of HMOs comprises, preferably consists essentially of 3′SL, 6′SL, LSTa, LSTb, LSTc, DSLNT, 2′FL, 3′FL, DFL, LNT, LNnT, LNFP I, LNFP II, LNFP III, LNFP V, LNDFH I, LNnDFH I and LNDFH II/LNnDFH II.

In a preferred aspect of the invention the synthetic mixture of HMOs comprises, preferably consists essentially of
(i) 1.4 to 2.6 wt % 3′-SL, more preferably 1.6 to 2.4 wt %, even more preferably 1.8 to 2.2 wt %;
(ii) 3.5 to 4.7 wt % 6′SL, more preferably 3.7 to 4.5 wt %, even more preferably 3.9 to 4.3 wt %;
(iii) 0.8 to 1.6 wt % LSTa, more preferably 0.9 to 1.5 wt %, even more preferably 1.0 to 1.4 wt %;
(iv) 0.7 to 1.4 wt % LSTb, more preferably 0.8 to 1.3 wt %, even more preferably 0.9 to 1.2 wt %;
(v) 4.1 to 4.8 wt % LSTc, more preferably 4.2 to 4.7 wt %, even more preferably 4.3 to 4.6 wt %;
(vi) 7.8 to 11.2 wt % DSLNT, more preferably 8.0 to 11.0 wt %, even more preferably 8.5 to 10.5 wt %;
(vii) 25 to 32 wt % 2′FL, more preferably 26 to 31 wt %, even more preferably 27 to 30 wt %;
(viii) 19 to 25 wt % 3′FL, more preferably 20 to 24 wt %, even more preferably 20.5 to 23 wt %;
(ix) 4.1 to 4.8 wt % DFL, more preferably 4.2 to 4.7 wt %, even more preferably 4.3 to 4.6 wt %;
(x) 5 to 10 wt % LNT, more preferably 6 to 9 wt %, even more preferably 7 to 8 wt %;
(xi) 0.5 to 1.1 wt % LNnT, more preferably 0.6 to 1 wt %, even more preferably 0.7 to 0.9 wt %;
(xii) 2.9 to 3.6 wt % LNFP I, more preferably 3 to 3.5 wt %, even more preferably 3.1 to 3.4 wt %;
(xiii) 3.8 to 4.4 wt % LNFP II, more preferably 3.9 to 4.3 wt %, even more preferably 4 to 4.2 wt %;
(xiv) 2.9 to 3.6 wt % LNFP III, more preferably 3 to 3.5 wt %, even more preferably 3.1 to 3.4 wt %;
(xv) 0.2 to 0.5 wt % LNFP V, more preferably 0.25 to 0.45 wt %, even more preferably 0.3 to 0.4 wt %;
(xvi) 4.2 to 5 wt % LNDFH I, more preferably 4.4 to 4.8 wt %, even more preferably 4.5 to 4.75 wt %;
(xvii) 0.00001 to 0.02 wt % LNnDFH I, more preferably 0.0001 to 0.015 wt %, even more preferably 0.001 to 0.01 wt %; and
(xviii) 0.7 to 1.3 wt of the combination of LNDFH II and LNnDFH II, more preferably 0.8 to 1.2 wt %, even more preferably 0.9 to 1.15 wt %;
based on total dry weight of the HMO mixture.

Alternatively worded, when the synthetic mixture of HMOs is part of a nutritional composition, said composition preferably comprises the following amounts of HMOs:
(i) 8 to 16 mg per 100 ml of 3′-SL, more preferably 9 to 15 mg per 100 ml, even more preferably 10 to 14 mg per 100 ml;
(ii) 20 to 30 mg per 100 ml of 6′SL, more preferably 22 to 26 mg per 100 ml, even more preferably 23 to 25 mg per 100 ml;
(iii) 4 to 10 mg per 100 ml of LSTa, more preferably 5 to 9 mg per 100 ml, even more preferably 6 to 8 mg per 100 ml;
(iv) 3 to 9 mg per 100 ml of LSTb, more preferably 4 to 8 mg per 100 ml, even more preferably 5 to 7 mg per 100 ml;
(v) 20 to 32.5 mg per 100 ml of LSTc, more preferably 22.5 to 30 mg per 100 ml, even more preferably 25 to 27.5 mg per 100 ml;
(vi) 50 to 60 mg per 100 ml of DSLNT, more preferably 52 to 56 mg per 100 ml, even more preferably 53 to 55 mg per 100 ml;
(vii) 125 to 200 mg per 100 ml of 2′FL, more preferably 140 to 185 mg per 100 ml, even more preferably 150 to 175 mg per 100 ml;
(viii) 100 to 150 mg per 100 ml of 3′FL, more preferably 110 to 140 mg per 100 ml, even more preferably 120 to 130 mg per 100 ml;
(ix) 38 to 50 mg per 100 ml of DFL, more preferably 40 to 48 mg per 100 ml, even more preferably 42 to 46 mg per 100 ml;
(x) 14 to 26 mg per 100 ml of LNT, more preferably 16 to 24 mg per 100 ml, even more preferably 18 to 22 mg per 100 ml
(xi) 3.5 to 5.5 mg per 100 ml of LNnT, more preferably 4 to 5 mg per 100 ml, even more preferably 4.5 to 5.5 mg per 100 ml;
(xii) 14 to 24 mg per 100 ml of LNFP I, more preferably 16 to 22 mg per 100 ml, even more preferably 18 to 20 mg per 100 ml;
(xiii) 18 to 30 mg per 100 ml of LNFP II, more preferably 20 to 28 mg per 100 ml, even more preferably 22 to 26 mg per 100 ml;
(xiv) 14 to 24 mg per 100 ml of LNFP III, more preferably 16 to 22 mg per 100 ml, even more preferably 18 to 20 mg per 100 ml;
(xv) 1.7 to 2.3 mg per 100 ml of LNFP V, more preferably 1.8 to 2.2 mg per 100 ml, even more preferably 1.9 to 2.1 mg per 100 ml;
(xvi) 20 to 3 mg per 100 ml of LNDFH I, more preferably 23 to 31 mg per 100 ml, even more preferably 25 to 29 mg per 100 ml;
(xvii) 0.00001 to 0.02 mg per 100 ml of LNnDFH I, more preferably 0.0001 to 0.015 mg per 100 ml, even more preferably 0.001 to 0.01 mg per 100 ml; and
(xviii) 2 to 10 mg per 100 ml of the combination of LNDFH II and LNnDFH II, more preferably 4 to 8 mg per 100 ml, even more preferably 5 to 7 mg per 100 ml;
based on volume of a nutritional composition comprising the HMO mixture according to the invention.

### Non-Digestible Saccharides

In a preferred embodiment, the HMO mixture may be provided together with a group II of non-digestible saccharides comprising oligo- and/or polysaccharides that preferably comprises at least two non-digestible oligosaccharides and/or polysaccharides, in particular two different sources of non-digestible oligosaccharides and/or polysaccharides. The non-digestible oligosaccharides of group II were found to provide further beneficial effects on prevention and/or treatment of infections.

Preferably the group II non-digestible saccharide is soluble. The term “soluble” as used herein, when having reference to a polysaccharide, fibre or oligosaccharide, means that the substance is at least soluble according to the method described by L. Prosky et al., J. Assoc. Off. Anal. Chem. 71, 1017-1023 (1988).

In one embodiment, the group II non-digestible saccharide is preferably selected from the group consisting of fructo-oligosaccharides (such as inulin), non-digestible dextrins, galacto-oligosaccharides (such as transgalacto-oligosaccharides), xylo-oligosaccharides, arabino-oligosaccharides, arabinogalacto-oligosaccharides, gluco-oligosaccharides, gentio-oligosaccharides, glucomanno-oligosaccharides, galactomanno-oligosaccharides, mannan-oligosaccharides, isomalto-oligosaccharides, nigero-oligosaccharides, chito-oligosaccharides, soy oligosaccharides, uronic acid oligosaccharides, and mixtures thereof.

In one embodiment, the group II non-digestible saccharide preferably includes a mixture of non-digestible poly- and/or oligosaccharides. The non-digestible oligosaccharide is preferably selected from the group consisting of fructo-oligosaccharide and galacto-oligosaccharide.

In one embodiment, the non-digestible saccharide is preferably selected from the group consisting of beta-galacto-oligosaccharide, alpha-galacto-oligosaccharide and galactan. According to a more preferred embodiment the non-digestible oligosaccharide is beta-galacto-oligosaccharide. Preferably the non-digestible oligosaccharide comprises galacto-oligosaccharide with beta-(1,4), beta-(1,3) and/or beta-(1,6) glycosidic bonds and a terminal glucose. Transgalacto-oligosaccharide is for example available under the trade name Vivinal©GOS (Domo FrieslandCampina Ingredients), Bi2muno (Clasado), Cup-oligo (Nissin Sugar) and Oligomate55 (Yakult). These oligosaccharides are thought to have a superior effect in decreasing the inflammatory response in nasal epithelial cells.

In a preferred embodiment, the galacto-oligosaccharide has an average DP in the range of 2-10, preferably 2-7, preferably 3-6.

In one embodiment, the non-digestible saccharide preferably comprises fructo-oligosaccharide. A fructo-oligosaccharide may in other context have names like fructopolysaccharide, oligofructose, polyfructose, polyfructan, inulin, levan and fructan and may refer to oligosaccharides comprising beat-linked fructose units, which are preferably linked by beta-(2,1) and/or beta-(2,6) glycosidic linkages, and preferably have a DP between 2 and 200. Preferably, the fructo-oligosaccharide contains a terminal beta-(2,1) glycosidic linked glucose. Preferably, the fructo-oligosaccharide contains at least 7 beta-linked fructose units. In a further preferred embodiment inulin is used. Inulin is a type of fructo-oligosaccharide wherein at least 75% of the glycosidic linkages are beta-(2,1) linkages. Typically, inulin has an average chain length between 8 and 60 monosaccharide units. A suitable fructo-oligosaccharide for use in the compositions according to the method or use of the present invention is commercially available under the trade name Raftiline*HP (Orafti). Other suitable sources are Raftilose (Orafti), Fibrulose and Fibruline (Cosucra) and Frutafit and Frutalose (Sensus).

In a preferred embodiment, the fructo-oligosaccharide has an average DP in the range of 7-100, preferably 11-60, preferably 20-50. Such long chain FOS (IcFOS) are preferred since they are believed to provide beneficial effects on amongst others the microbiota and stool consistency and are believed to substitute some of the functions of long-chain HMOs, i.e. those having a DP of 7 and higher.

In one embodiment, the group II saccharides are a mixture of galacto-oligosaccharide and fructo-oligosaccharide. Preferably the mixture of galacto-oligosaccharide and fructo-oligosaccharide is present in a weight ratio of from 1/99 to 99/1, more preferably from 1/19 to 19/1, more preferably from 1/1 to 19/1, more preferably from 2/1 to 15/1, more preferably from 5/1 to 12/1, even more preferably from 8/1 to 10/1, even more preferably in a ratio of about 9/1. This weight ratio is particularly advantageous when galacto-oligosaccharide has a low average DP and fructo-oligosaccharide has a relatively high DP.

In a preferred aspect, the total weight ratio of both the mixture of HMOs and the group II non-digestible saccharides of short chain oligosaccharides having a DP between 3 and 6 (DP 3-6) to long chain oligosaccharides have a DP of 7 and higher is from 1/99 to 99/1, more preferably from 1/19 to 19/1, more preferably from 1/1 to 19/1, more preferably from 2/1 to 15/1, more preferably from 5/1 to 12/1, even more preferably from 8/1 to 10/1, even more preferably in a ratio of about 9/1.

In a preferred aspect a nutritional composition is provided that comprises scGOS, IcFOS and the mixture of HMOs and wherein the total ratio of short chain (DP 3-6) to long chain oligosaccharides of scGOS, IcFOS and HMOs is about 9:1.

Without being bound by theory it is believed that the provision of a balanced mixture of monovalent short chain oligosaccharides and multivalent long chain saccharides provides for beneficial effects on the immune system and an optimal effect in terms of prevention and treatment of infections.

Most preferred is a mixture of galacto-oligosaccharide with an average DP below 10, preferably below 6, preferably in the range of 2-10, preferably in the range of 2-7, preferably in the range of 3-6, and fructo-oligosaccharide with an average DP above 7, preferably above 11, even more preferably above 20, preferably in the range of 7-100, preferably in the range of 11-60, preferably in the range of 20-50.

Preferably a composition according to the present invention comprises 2.5 to 20 wt % total non-digestible oligosaccharide, more preferably 2.5 to 15 wt %, even more preferably 3.0 to 10 wt %, most preferably 5.0 to 7.5 wt %, based on total dry weight of the composition, i.e., the total wt % of both the mixture of HMOs and the group II non-digestible oligosaccharides. When in liquid form, the composition in the present method or use preferably comprises 0.35 to 2.5 wt % total non-digestible oligosaccharide, more preferably 0.35 to 2.0 wt %, even more preferably 0.4 to 1.5 wt %, based on 100 ml of the composition.

### Nutritional Composition

The HMO mixture according to the invention is preferably provided in a nutritional composition, preferably in the form an infant formula, a follow-on formula, or a young child formula. Likewise, the HMO mixture according to the invention and the group II of non-digestible oligosaccharide is preferably provided in a nutritional composition, preferably in the form an infant formula, a follow-on formula, or a young child formula. The nutritional composition according to the invention is not native cow's milk or native milk from another mammal. The present nutritional composition preferably comprises digestible carbohydrates, protein, and lipid, wherein the lipid preferably provides 30 to 60%, preferably 35 to 55% of the total calories, the protein provides 5 to 15%, more preferably 6 to 12%, even more preferably 7 to 9% of the total calories and the digestible carbohydrates provide 25 to 75%, more preferably 40 to 60% of the total calories. Non-digestible oligosaccharides have a caloric density of 2 kcal/g and preferably make up 0.4 to 7% of total calories. The nutritional composition preferably comprises 3 g to 7 g lipid/100 kcal, preferably 4 g to 6 g lipid/100 kcal, more preferably 4.5 g to 5.5 g lipid/100 kcal; it preferably comprises 1.25 g to 4 g protein/100 kcal, more preferably 1.5 g to 3.0 g protein/100 kcal, more preferably 1.8 g to 2.2 g protein/100 kcal and it preferably comprises 6 g to 20 g digestible carbohydrate/100 kcal, more preferably 10 g to 15 g digestible carbohydrate/100 kcal.

Preferably the nutritional composition has an energy density of 45 to 75 kcal/100 ml, more preferably 60 to 70 kcal/100 ml, even more preferably 65 to 70 kcal/100 ml when in a ready-to-use form. This density ensures an optimal balance between hydration and caloric intake. The osmolarity of the present composition is preferably between 150 and 420 mOsmol/I, more preferably 260 to 320 mOsmol/I.

The nutritional composition is preferably a solid product, preferably a powder. Suitably, the nutritional composition is in a powdered form, which can be reconstituted with water, to form a ready-to-use liquid. Alternatively, the nutritional composition may be a ready-to-use liquid or is in a liquid concentrate form that should be diluted with water to a ready-to-use liquid.

The nutritional composition preferably comprises digestible carbohydrates. Based on calories the nutritional composition preferably comprises 6 g to 20 g digestible carbohydrates per 100 kcal, more preferably 10 g to 15 g per 100 kcal. When in liquid form, e.g., as a ready-to-use liquid, the nutritional composition preferably comprises 4 g to 15 g digestible carbohydrate per 100 ml, more preferably 7 g to 10 g per 100 ml. Based on dry weight the nutritional composition preferably comprises 30 wt % to 85 wt %, more preferably 40 wt % to 65 wt % digestible carbohydrates. Worded alternatively, when the nutritional composition is in powder form, the digestible carbohydrates are preferably present in an amount of 40 g to 85 g/100 g dry weight, more preferably 40 g to 65 g/100 g dry weight. Preferred digestible carbohydrate sources are one or more of lactose, glucose, sucrose, fructose, galactose, maltose, starch, and maltodextrin. Lactose is the main digestible carbohydrate present in human milk. Lactose advantageously has a low glycemic index. The nutritional composition preferably comprises lactose. The nutritional composition preferably comprises digestible carbohydrate, wherein at least 35 wt %, more preferably at least 50 wt %, more preferably at least 75 wt %, even more preferably at least 90 wt %, most preferably at least 95 wt % of the digestible carbohydrate is lactose.

The nutritional composition preferably comprises protein. The protein concentration in the nutritional composition is determined by the sum of protein, peptides, and free amino acids. Preferably the nutritional composition comprises 1.25 g to 4 g protein per 100 kcal, even more preferably 1.5 g to 3.0 g protein per 100 kcal, even more preferably 1.8 to 2.2 g per 100 kcal. A low protein concentration advantageously is closer to human milk as human milk comprises a lower amount of protein based on total calories compared to cow's milk. Based on a ready-to-use liquid product the nutritional composition preferably comprises 0.8 to 2.5 g per 100 ml, more preferably 1.0 g to 2.0 g/100 ml, even more preferably 1.2 to 1.5 g per 100 ml. Based on dry weight the nutritional composition preferably comprises 6 to 18 wt %, more preferably 7 to 15 wt %, even more preferably 8 to 11 wt % protein. Worded alternatively, when the nutritional composition is in powder form, the proteins are preferably present in an amount of 6 g to 18 g/100 g dry weight, more preferably 7 to 15 g, even more preferably 8 to 11 g/100 g dry weight of the composition. The source of the protein is preferably selected in such a way that the minimum requirements for essential amino acid content are met, and satisfactory growth is ensured. Hence protein sources based on cows' milk proteins such as whey, casein, and mixtures thereof and proteins based on soy, rice or pea are preferred. In case whey proteins are used, the protein source is preferably based on acid whey or sweet whey, modified sweet whey, whey protein isolates or mixtures thereof.

The nutritional composition of the present invention preferably comprises lipids. The lipid is preferably present in an amount of 3 g to 7 g per 100 kcal, more preferably in an amount of 4 g to 6 g lipid per 100 kcal and most preferably in an amount of 4.5 g to 5.5 g lipid per 100 kcal. When in liquid form, e.g., as a ready-to-use liquid, the nutritional composition preferably comprises 2.2 g to 4.5 g lipid per 100 ml, more preferably 2.5 to 4.0 g, even more preferably 3.0 to 3.75 g per 100 ml. Based on dry weight the nutritional composition preferably comprises 16 to 32 wt %, more preferably 18 to 30 wt %, even more preferably 20 to 28 wt % lipid. Worded alternatively, when the nutritional composition is in powder form, the lipids are preferably present in an amount of 16 g to 32 g/100 g dry weight, more preferably 18 to 30 g, even more preferably 20 to 28 g/100 g dry weight of the composition.

The lipid preferably comprises vegetable lipid. The presence of vegetable lipid advantageously enables an optimal fatty acid profile high in polyunsaturated fatty acids, such as essential linolenic acid and alpha-linolenic acid and is more reminiscent to human milk fat. Lipid from non-human mammalian milk alone, e.g., cow milk, does not provide an optimal fatty acid profile. The amount of essential fatty acids is too low in non-human mammalian milk. Preferably the nutritional composition comprises at least one, preferably at least two vegetable lipid sources selected from the group consisting of linseed oil (flaxseed oil), rape seed oil (such as colza oil, low erucic acid rape seed oil and canola oil), sunflower oil, high oleic sunflower oil, safflower oil, high oleic safflower oil, olive oil, coconut oil, soy oil, palm oil and palm kernel oil.

Additionally animal fat such as cow's milk fat is preferably present in the nutritional composition. Such sources of lipid may provide additional desired components such as butyric (BA) and caproic acid (CA) and beta-palmitic acid (sn2-PA). Components such as butyric acid are known to have a synergistic effect together with human milk oligosaccharides on the gut barrier, immune system, and anti-pathogenic effect. Preferably the nutritional composition comprises at least 0.5 wt % butyric acid based on total fatty acids, more preferably at least 0.7 wt % to 2 wt %.

Additionally, egg oil and/or fish oil and/or microbial oils such as oil from fungi and algae may be present. Such oils are suitable sources of long-chain polyunsaturated fatty acids such as docosahexaenoic acid (DHA), arachidonic acid (ARA) and/or eicosapentaenoic acid (EPA). Preferably the nutritional composition comprises n3 LC-PUFA, such as EPA and/or DHA, more preferably DHA. Preferably the nutritional composition comprises at least 0.05 wt %, preferably at least 0.1 wt %, more preferably at least 0.2 wt %, DHA based on total fatty acids. Preferably the nutritional composition comprises not more than 2.0 wt %, preferably not more than 1.0 wt % DHA based on total fatty acids. The nutritional composition preferably comprises ARA. Preferably the nutritional composition comprises at least 0.05 wt %, preferably at least 0.1 wt %, more preferably at least 0.2 wt % ARA based on total fatty acids.

Preferably further ingredients are present in the nutritional composition like vitamins, minerals, trace elements, nucleotides and other micronutrients as known in the art.

In some aspects, the nutritional composition may comprise lactic acid producing bacteria selected from the group consisting of the genera Bifidobacterium and/or Lactobacillus, in particular Bifidobacterium. The intestinal microbiota of breastfed infants is high in bifidobacteria. Addition to the nutritional composition of one or more strains belonging to the Bifidobacterium genus will further improve the intestinal microbiota and its activity, and therewith have additional advantageous effects on pathogenic infections. More preferably, the nutritional composition comprises Bifidobacterium breve, Bifidobacterium longum spp. Iongum, Bifidibacterium longum spp infantis and/or Bifidobacterium bifidum. Such strains of the species of Bifidobacterium are commercially available or can be isolated from the microbiota of infants. The amount of Bifidobacterium and/or Lactobacillus is preferably 10⁴ to 10¹¹ cfu per gram dry weight of the nutritional composition.

The synthetic mixture of HMOs according to the invention is preferably provided in a nutritional composition in the form of an infant formula, a follow-on formula, or a young child formula. The nutritional composition can be advantageously applied as a complete nutrition for infants. This means that the composition that is to be administered is not human milk. Infant formula or follow-on formula or young child formula means that it concerns a composition that is artificially made or in other words that it is a synthetic composition. In the context of the present invention, young child formula can also be named growing-up milk. The present nutritional composition preferably is intended for, or is used for, providing nutrition to an infant or young child,

Infant formulas are intended for infants from birth to about 4 to 6 months of age and are intended as a substitute for human milk. Typically, infant formulae are suitable to be used as sole source of nutrition. Such infant formulae are also known as starter formula. In the context of the present invention this is referred to as a nutritional composition, or infant formula, for the first 6 months of life.

Follow-on formulas are intended for infants starting from 4 to 6 months of age to 12 months of age and are intended to be supplementary feedings for infants that start weaning on other foods. In the context of the present invention this is referred to as a nutritional composition, or follow-on formula, for the age of 6 to 12 months.

Young child formula refers to nutritional compositions, artificially made, intended for children of 12 months up to and including 47 months, in other words for children of 1 to 3 years of age which are intended to be supplementary feedings. In the context of the present invention this is referred to as a nutritional composition, or young child formula, for the age of 12 to 47 months, for the age of 1 to 3 years.

Infant formulae and follow-on formulae are subject to strict regulations, for example for the EU regulations no. 609/2013 and no. 2016/127 and Codex Alimentarius for Infant Formula, CODEX STAN 72-1981. Young child formulas preferably follow the directive for follow-on formula.

The nutritional composition is preferably an infant formula or a follow-on formula.

Looking at human milk, the amount of HMOs reduces during lactation with increasing age of the infant. In a preferred embodiment an infant formula comprises more HMOs than a same amount or volume of follow-on formula and a follow-on formula comprises more HMOs than a same amount or volume of young child formula. Since young children consume less formula as part of the total diet than infants where infant formula may form the sole source of nutrition it is in some embodiment preferred to provide increased amounts of HMO in a young child formula to provide for a desired daily amount of HMOs

### Application

The HMO mixture and nutritional composition comprising said HMO mixture of the present invention are preferably used for providing nutrition to an infant or young child, preferably an infant.

Preferably the nutritional composition of the present invention is provided to a human subject during the first 3 years of life. Preferably, the nutritional composition is used in a method or for use for providing nutrition to a human subject in the first 12 months of life, optionally the first 3 years of life. In an aspect the nutritional composition comprising the HMO mixture is a first infant formula for the first 6 months of life, wherein the formula comprises the mixture of HMOs according to the invention.

The invention particularly concerns the use of the HMO mixture for preventing and/or treating infections, preferably pathogenic infections. The invention further pertains to reducing the risk at infections, preferably pathogenic infections. The invention further pertains to the use of the HMO mixture for preventing and/or treating pathogenic infections, preferably viral and/or bacterialinfections in a human subject, more preferably at least viral and bacterial infections. The HMO mixture comprises a plurality of different HMOs providing a variety of properties and biological activities including effects on pathogen binding, modulation of cell binding of pathogens and modulation of immune responses. Advantageously the mixture of HMOs prevents and/or treats more than a single pathogenic infection, i.e., the mixture advantageously provides prevention and/or treatment to a broad range of pathogenic infections. The synthetic mixture of HMOs further advantageously prevents resistance to the prevention and/or treatment of pathogenic infections. By the provision of a broad range of HMO structures pathogenic evasion of specific HMOs is prevented. The HMO mixture thus advantageously provides long-term prevention and/or treatment of pathogenic infections.

Without wishing to be bound by theory it is believed that the synthetic mixture of HMOs according to the invention is more resilient to the evolution of microbial pathogenicity. In this way the HMO mixture will be future proof and provide long-term beneficial effects being resistant to mutations of the pathogen. In addition, it is believed that the provision of a broad spectrum of HMOs also allows for the provision of a mixture that supports the need of genetically diverse subjects with different susceptibilities and responses to the HMO structures.

In an aspect the invention concerns a synthetic mixture of HMOs and nutritional compositions comprising the HMO mixture for preventing and/or treating infections, preferably pathogenic infections, wherein the prevention and/or treatment of infections is improved as compared to the prevention and/or treatment of infections observed with oligosaccharide mixtures of less than 6 different oligosaccharides. In a preferred embodiment the prevention and/or treatment of infections is improved as compared to the prevention and/or treatment of infections observed with oligosaccharide mixtures of less than 6 different oligosaccharides including 2′-FL, GOS and FOS, and/or a mixture of 5 HMOs.

In a further aspect the invention concerns a HMO mixture and nutritional compositions comprising the HMO mixture for the prevention and/or treatment of infections, preferably pathogenic infections, wherein the prevention and/or treatment of infections is comparable and/or improved in comparison to the prevention and/or treatment of infections with HMOs of human breast milk. The effect of the HMO mixture and nutritional compositions comprisingthe HMO mixture is thus an effect at least closer to human breast milk.

In an aspect the invention concerns a non-therapeutic use and method comprising administering the HMO mixture and nutritional compositions comprising the HMO mixture for improving resistance against infections, preferably pathogenic infections, in human subject, preferably healthy and/or term infants.

In a further preferred aspect, the invention concerns an HMO mixture and nutritional compositions comprising the HMO mixture for use in therapy. The invention concerns therapeutic uses wherein the human subject is preferably selected from preterm infants, (very) low birth weight infants, C-section born infants and antibiotic treated infants. The latter infants have a disturbed microbiota and are particularly vulnerable for pathogenic infections.

In a preferred aspect the HMO mixture nutritional compositions comprising the HMO mixture is for use in the prevention and/or treatment of infection. More preferably the use in the prevention and/or treatment of pathogenic infections comprises prevention and/or treatment of at least viral and/or bacterial pathogenic infections.

In a particular aspect the HMO mixture is for use in the prevention and/or treatment of infection is with a virus selected from double-stranded RNA viruses, single-stranded positive and negative sense RNA viruses, and DNA viruses and/or bacteria selected from gram-positive and gram-negative bacteria. In an aspect the pathogenic bacteria are selected from the Listeriaceae, Enterobacteriaceae and Staphylococcus family.

In a further particular aspect, the HMO mixture is for use in the prevention and/or treatment of infections, preferably pathogenic infections by a virus selected from rotavirus, respiratory syncytial virus (RSV) and SARS-COV2 and/or bacteria selected from L. monocytogenes, E. coli, S. aureus, S. typhimurium, L. monocytogenes, E. faecium, E. faecalis, P. mirabilis and K. pneumoniae, preferably E. coli.

In other words, the invention thus concerns the use of the HMO mixture in the manufacture of a nutritional composition for prevention and/or treatment of infections, preferably pathogenic infections in a human subject.

For some jurisdictions this can also be worded as the use of the HMO mixture for improving resistance against infections, preferably pathogenic infections, in particular non-therapeutically improving resistance against infections of infections, preferably pathogenic infections, in a human subject, preferably a healthy, term infant or young child.

### EXAMPLES

### Example 1: Mixture of HMOs

An exemplary HMO mixture is provided.

| HMOs | [g/L]¹ |
|---|---|
| 2′-FL | 1.65 |
| 3′-FL | 1.24 |
| 3′-SL | 0.12 |
| 6′-SL | 0.24 |
| DFL | 0.20 |
| LNT | 0.44 |
| LNnT | 0.05 |
| LNFP I | 0.19 |
| LNFP II | 0.24 |
| Lacto-N-fucopentaose II | |
| LNFP III | 0.19 |
| Lacto-N-fucopentaose III | |
| LNFP V | 0.02 |
| Lacto-N-fucopentaose V | |
| LSTa (acidic) | 0.07 |
| LSTb (acidic) | 0.06 |
| LSTc (acidic) | 0.26 |
| LNDFH I | 0.27 |
| Lacto-N-difucohexaose I (Le b) | |
| LNnDFH I | 0.0001 |
| Lacto-N-neodifucohexaose I | |
| LNDFH II/LNnDFH II | 0.06 |
| Lacto-N-difucohexaose II (Le a)/ | |
| Lacto-N-neodifucohexaose*II (Le x) | |
| DSLNT | 0.54 |
| Disialyllacto-N-tetraose | |
| total | 5.84 |

*synonymous to: LNDFH III

### Test Compounds

The test compounds use in examples 2 to 5 are according to the following specifications:
The total HMOs fraction represents all HMOs which can be found in mature human milk in the respective weight ratio's as observed in mature human breast milk and ranging in DP between 3 and 28 or higher. This HMO fraction was yielded by depleting the Lactose and mineral content from a total carbohydrate mineral fraction extracted from mature pooled human milk (see Chia L W, Mank M, et al. Cross-feeding between Bifidobacterium infantis and Anaerostipes caccae on lactose and human milk oligosaccharides. Benef Microbes. 2021; 12(1):69-83.).

The synthetic mix of 19 HMOs was created by re-combining defined individual neutral and acidic HMO fractions ranging between a degree of polymerisation (DP) of 3-6. These acidic and neutral HMO fractions were also yielded by preparative SEC separation from a human milk total carbohydrate and mineral fraction as described in Chia et al. The 19 HMO mixture used was prepared according to amounts of the mixture of example 1 described above.

For the mixture of GOS:FOS at a ratio of 9:1 scGOS was derived from Vivinal GOS and the IcFOS was derived from RaftilineHP. 5 HMOs: a mixture of 48.5% 2′-fucosyllactose, 11.6% 3-fucosyllactose, 26.0% lacto-N-tetraose, 4.5% 3′-sialyllactose, and 5.2% 6′-sialyllactose was obtained from Chr. Hansen HMO GmbH, Rheinbreitbach, Germany.

### Example 2: Effect of HMO Mixture on SARS-CoV-2 Infection

To assess the potential of the 19 HMO mixture according to example 1 to either neutralize or provide anti-infective effects to an infection with a positive-sense single-stranded RNA virus such as SARS-CoV2 WA1 (USA-WA1/2020 isolate; BEI Resources, cat #NR-52281) human lung epithelial carcinoma cells (A549-hACE2/TMPRSS2; Invivogen, a549-hace2tpsa) genetically engineered to overexpress the primary entry receptor of SARS-CoV-2 angiotensin-converting enzyme 2 (ACE2) along with its entry cofactor, the membrane-bound protease TMPRSS2, were cultured according to the following experimental protocol:
Virus pre-incubation protocol: Pre-incubation of SARS-CoV2 WA1 with HMOs prior to infection of A549-hACE2/TMPRSS2 cells.

### Cell Seeding

A549-hACE2/TMPRSS2 cells were seeded in 96 well plates (Costar, 3610) in culture medium (DMEM Glutamax, 10% FBS, 100 U/mL penicillin-streptomycin, 0.5 μg/mL Puromycine, 300 μg/mL hygromycine B, 100 mM sodium pyruvate) and incubated at 37° C. and 5% Co₂ overnight.

### Test Compounds

HMO mixtures were prepared in sterile water to a stock solution of 16 mg/ml, then serially diluted 2-fold in infection medium (DMEM Glutamax, 2% FBS, 100 U/mL penicillin-streptomycin) to obtain a working dilution of 16, 8, 4, 2, 1, and 0.5 mg/ml.

The following compounds were assessed in the 6 concentrations mentioned above and in triplicate for their capacity to inhibit SARS-CoV2 infection:
2′-FL
A mixture of GOS:FOS at a 9:1 weight ratio
Total HMOs
Mixture of 19 HMOs
6′-SL
Untreated control

### Virus Incubation

Test compounds were transferred to the virus containing plates and additional infection medium was added to ensure a test concentration range of 0.5-16 mg/mL. The plates were incubated at 37° C. and 5% CO₂ for 1 hour.

### Infection of A549-hACE2/TMPRSS2Cells

Culture medium was removed from A549-hACE2/TMPRSS2 cell plates and replaced with HMO-treated SARS-CoV-2 to achieve a multiplicity of infection (MOI) of 0.1. Plates were incubated at 37° C. and 5% CO₂ for 1 h. Culture medium was then removed from plates; all wells were washed once with 1×PBS then replenished with 100 μl of fresh medium. Plates were incubated for 48 h at 37° C., 5% CO₂.

### Determination of Viral Inhibition

To assess the efficacy of the different test compounds to inhibit viral infection, viral RNA (vRNA) was quantified by RT-qPCR with a Taqman Fast Virus 1-step kit (Thermo Fisher Scientific, 4444434) using oligos and probes within the N gene of SARS-CoV-2 (IDT; 10006830, 10006831 and 10006832). Data analysis was done using GraphPad Prism v9.1 wherein the data was calculated as percentage of the untreated control and then normalized to the effect of the lowest compound concentration.

### Results

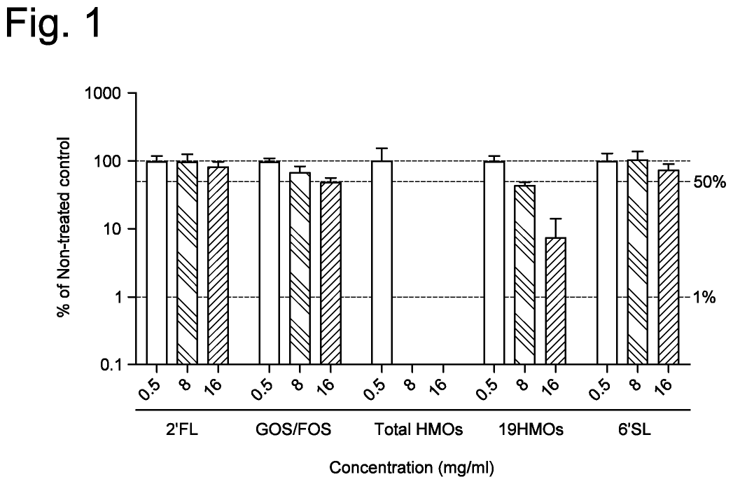

A decrease in viral RNA correlates with a decrease in presence of virally infected cells. 2′-FL, and 6′-SL did not show viral neutralization efficacy upon re-incubation of SARS-CoV2 WA1 with these HMOs over the range of concentrations assessed. GOS:FOS as well as the mixture of 19 HMOs showed viral neutralization efficacy after pre-incubation at higher concentrations, while the total HMO mixture showed strong antiviral activity already at lower concentrations (FIG. 1).

Overall, the mixture of 19 HMOs was found to be effective in neutralizing viruses with an effect comparable to the mixture of total HMOs and improved compared to the single fucosylated or sialylated oligosaccharides.

### Example 3: Effect of HMO Mixture on Rotavirus Infection

To assess the effect of the mixture of HMOs on infection with a double-stranded RNA virus, the Caco-2 epithelial cell barrier model was used to assess the effects of HMOs on infection with Rotavirus in vitro.

Caco-2 cells were used according to established methods. In brief: cells were cultured in MEM (Minimum Essential Medium), supplemented with 10% FCS heat inactivated, 100 units/ml penicillin, 0.1 mg/ml streptomycin, 2 mM sodium pyruvate and non-essential amino acids, and seeded at a density of 0.3×10⁵ cells into 1.13 cm² ThinCert insert, a polyethylene terephthalate membrane (Greiner, Monroe, North Carolina, USA) with 0.4 μm pore density, and placed in a 12-well plate. The Caco-2 cells were maintained in a humidified atmosphere of 95 percent air and 5 percent CO₂ at 37 degrees centigrade. The transepithelial electrical resistance (TEER) was measured as a quantitative marker for barrier integrity. After 3 weeks culturing, a confluent monolayer was obtained with a mean Transepithelial electrical resistance (TEER) exceeding 400 W cm² measured by a Millicell-Electrical Resistance System voltohm-meter (Millipore, Temecula, CA, USA).

After culturing for 3 weeks, the cells are well differentiated and form a barrier layer on the ThinCert inserts. Cells were subsequently incubated with the different test compounds (namely: a mixture of 19 HMO, a mixture of 5 HMO, medium only, lactose, cellobiose or lactoferrin for 4 h in serum free culture medium and at a concentration of 50 mg/ml after that the rotavirus (RV) strain SA11 at MOI 0.1 was added.

The next day, 20 hour post infection (p.i.) measurements of the transepithelial electrical resistance (TEER) and lucifer yellow (LY) permeability were conducted to investigate barrier integrity. For TEER measurements a Millicel-ERS voltohm-meter connected to a pair of chopsticks electrodes was used to measure the TEER values. For paracellular tracer flux assay the membrane impermeable lucifer yellow (LY) (Sigma, St Luis, MO, USA) was added to allow for the assessment of how leaky the barrier has become. Lucifer yellow was added in a concentration of 32 ug/ml to the apical compartment in the ThinCert plate for 48 h, and the paracellular flux was determined by measuring the fluorescence intensity in the basolateral compartment with a spectrophotofluorometer (FlexStation 3, Molecular Devices, San Jose, CA, USA) set at excitation and emission wavelengths of 410 and 520 nm, respectively. The diffusion of LY was evaluated 48 h p.i. (p.i., post infection; RV, rotavirus; LY, Lucifer yellow).

### Results and Conclusions

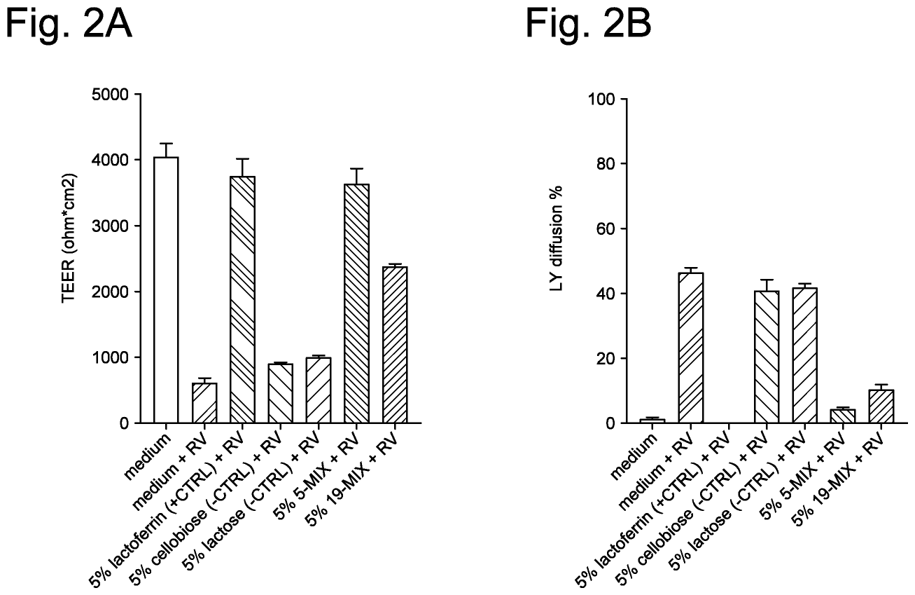

Exposure to 5% of a mixture of 5 HMO almost completely prevented the rotavirus induced decrease in TEER at 20 h p.i suggestive of an infection-preventive effect (FIG. 2A). Exposure to 5% of a mixture of 19 HMO showed a similar preventive effect, with a lower magnitude reflective of the slightly lower concentration of the 5HMO structures present in the 19 HMO mix. The use of a different control sugar such as 5% Lactose or cellobiose (having no effect) shows that the observed effects are specific for the oligosaccharides in the 5 HMO and 19 HMO mix. The results on the barrier leakiness (FIG. 2B) show that preincubating Caco-2 cells with 5% of 5HMO and 5% 19 HMO prevents barrier disruption and the diffusion of LY to the basolateral compartment.

Overall, the data shows that the mixture of 19 HMOs according to the invention is effective against rotavirus infection.

### Example 4: Effect of HMOs on RSV Infection

To assess the effect of mixtures of HMOs on a negative-sense single-stranded RNA virus in vitro infection assays were performed with RSV-A2 strain. Pre-incubation of either HEp-2 cells or RSV-A2 with HMOs prior to infection was compared to assess anti-viral activity. Hep2 cells were exposed to the following conditions as discussed in more detail separately:
I. Pre-incubation of HEp-2 cells with HMOs before viral infection.
II. Pre-incubation of RSV-A2 with HMOs prior to infection of HEp-2 cells.

### I. HMO Pre-Incubation of Hep-2 Cells Followed by Viral Infection

### Cell Seeding

HEp-2 cells were seeded in white [Greiner 655098]96 well plates in assay medium (EMEM [Sigma M2279]) supplemented with 2% heat-inactivated fetal bovine serum (HI-FBS) [Gibco 10500064], 1% Penicillin/streptomycin [Gibco 15140122] and 1% L-glutamine [Gibco 25030024] and incubated at 37° C. and 5% CO₂ for 5 hours.

### Treatment of HEp-2 Cells

HMO mixtures were prepared in sterile water and ribavirin (compound that blocks viral RNA synthesis and viral mRNA capping) was prepared in 1% DMSO at 10× test concentration in a round-bottom 96-well plate [Corning 3788], then serially diluted 2-fold.

The following compounds were assessed for their capacity to inhibit RSV-A2 viral infection:
2′-FL
A mixture of GOS:FOS at a 9:1 weight ratio
Total HMOs
Mixture of 5 HMOs
Mixture of 19 HMOs
6′-SL

Test compounds were subsequently transferred to the HEp-2 cell plates. Additional assay medium was added to ensure a final HMO concentration range of 0.13-16 mg/mL. Cell plates were incubated for 24 hours at 37° cand 5% CO₂.

### Viral Infection

After 24-hour incubation with the test conditions RSV-A2 was added to the HMO containing HEp-2 cell plates to achieve a final multiplicity of infection [MOI] of 0.5. Cell plates were incubated for a further 72 hours at 37° C. and 5% CO₂.

### Determination of Viral Inhibition

Viral ToxGlo™ [Promega G8943] was added to the plates to assess cell metabolism following viral infection and HMO treatment. After 20 minutes incubation, luminescence of each plate was measured on a spectrophotometer. Data were analysed using GraphPad Prism software to determine the concentration effective in producing 50% of viral inhibition expressed as ECso values for ribavirin and HMOs where relevant.

### Results

The experiments were performed in duplicate. The percentage viral inhibition is shown in table 1. For some tested compounds the assessed concentration range was too low to accurately determine the ECso values, ECso values are shown for 2′-FL and the 19 HMO mixture.

In replicate 1, total HMOs, 2′-FL, as well as the 19 HMO mix inhibited infection when pre-incubated with HEp-2 cells. This suggests an effect of these HMOs on the cells prior to infection to prevent infection. While the mixture of GOS:FOS showed some very limited antiviral activity (<50% inhibition) in the first replicate, 6′-SL displayed no anti-viral activity at the concentration tested.

In replicate 2 a higher percentage of infection was achieved (94% in replicate 2 versus 67% in replicate 1, data not shown) leading to higher doses of the positive control ribavirin being needed to achieve ECso; In these conditions of higher percentage of infection, it was found that only the 19 HMO mixture showed beneficial inhibition of infection when pre-incubated with the HEp-2 cells. Notably, the 19 HMO mixture maintained its effect on inhibiting viral infection with >50% despite the higher percentage of infection in the second replicate (data not shown).

TABLE 2
Percentage viral inhibition achieved by pre-incubating
Hep-2 cells with different test compounds or mixtures.

| | | Test | |
|---|---|---|---|
| | | concentration | Absolute |
| | Maximum viral | maximum viral | EC50 |
| Test compound | inhibition (%) | inhibition (mg/mL) | (mg/ml) |
| 2′-FL | 78.8 | 16 | 12.85 |
| GOS:FOS | 39.70 | 8 | n/a |
| Total HMOs | 70.3 | 1 | n/a |
| 5 HMOs | 122 | 16 | 12.85 |
| 19 HMOs | 123.9 | 16 | 7.59 |
| 6′-SL | 2.6 | 8 | n/a |

II. Pre-Incubation of RSV-A2 with HMOs Prior to Infection of HEp-2 Cells

### Cell Seeding

HEp-2 cells were seeded as described above and incubated at 37° C., 5% CO₂ for 5 hours.

### Treatment of RSV-A2

HMOs and ribavirin were prepared at 10× test concentrations as described previously. RSV-A2 was diluted in assay medium and added to round-bottom 96-well plates [Corning 3788]. The following compounds were assessed for their capacity to inhibit RSV-A2 viral infection:
2′-FL
A mixture of GOS:FOS at a 9:1 weight ratio
Mixture of 5 HMOs
Mixture of 19 HMOs
6′-SL
Ribavirin (positive control for viral inhibition)

Test compounds were transferred to the virus containing plates and additional assay medium was added to ensure a test concentration range of 0.13-16 mg/mL. The plates were incubated at 37° C. and 5% CO₂ for 1 hour.

### Infection of HEp-2 Cells

Assay medium was removed from HEp-2 cell plates and replaced with HMO-treated RSV-A2 to achieve an MOI 0.5. Plates were incubated at 37° C. and 5% CO₂ for a further 72 hours.

### Determination of Viral Inhibition

Viral ToxGlo™ [Promega G8943] was added to the plates to assess cell metabolism following viral infection with untreated RSV-A2 control or HMO-pretreated RSV-A2. Following 20 minutes incubation, luminescence of each plate was measured on a spectrophotometer. Data were analyzed using GraphPad Prism software to generate ECso values for ribavirin and HMOs where relevant.

### Results

The experiments were performed in duplicate. The percentage viral inhibition is shown in table 3 for each experimental condition. For some tested compounds the assessed concentration range was too low to accurately determine the ECso values, ECso values are shown for GOS:FOS, and the 19 HMO mixture.

GOS:FOS, and the mixture of 19 HMOs showed antiviral activity when pre-incubated with RSV-A2 prior to infection. This was observed in both replicates, where a similar percentage of infection was achieved in the RSV-A2 control group (94-95%). 6′-SL displayed no anti-viral activity at the concentration tested, while also the 5 HMO mixture only exhibited a low degree of viral inhibition.

Overall, the mixture of 19 HMOs appears to have a broad effect on both neutralizing viruses and inhibition cell infection, similar to the effect of the total HMOs, but superior compared to the effects observed with the other oligosaccharides. Only 19HMOs mixture maintained its effect on inhibiting viral infection with >50% after Hep2 cell preincubation despite the higher percentage of infection in the second replicate.

TABLE 3
Percentage viral inhibition achieved by pre-incubating
RSV-A2 with different test compounds or mixtures.

| | | Test | |
|---|---|---|---|
| | | concentration | Absolute |
| | Maximum viral | maximum viral | EC50 |
| Test compound | inhibition (%) | inhibition (mg/mL) | (mg/ml) |
| 2′-FL | 2.88 | 16 | |
| GOS:FOS | 111.98 | 16 | 8.50 |
| 5 HMOs | 3.43 | 16 | n/a |
| 19 HMOs | 91.15 | 16 | 6.93 |
| 6′-SL | −0.58 | 1 | n/a |

### Example 5: Effect of HMOs on the Growth of Pathogenic Bacteria

The effect of the total HMO mixture, the mixture of 19 HMOs, a mixture of 5 HMOs and a mixture of total HMOs with lactose in a concentration between 0.5 mg/ml to 16 mg/ml to inhibit growth of gram-negative enterobacterium E. coli (table 4) was assessed by growth percentage determination.

E. coli inoculated in 5 ml of YCFA broth supplemented with glucose as the sole energy source (YCFAG) and incubated anaerobically at 37° C. for 24 h. Subsequently, the strain was propagated to 5 ml ofYCFAG broth culture overnight as pre-culture; after that, different strains were added to the tubes of YCFA broth supplemented with the different carbon sources at a ratio of 1:100 in duplicate at 37° C. Growth in cultures was monitored spectrophotometrically every 4 h from 0- to 72-h culture by measuring the OD600 by using Ultrospec 10 cell density meter (Amersham Biosciences GmbH, Germany).

The growth percentage was determined by assessment of the following 4 parameters with the total HMO fraction with lactose as negative control: i) ptMAX (A), ii) time till 50% OD600, iii) Max OD and iv) the area under the curve. A higher growth percentage is thus indicative for less or no growth inhibition and vice versa.

Overall, the mixture of 19 HMOs was found effective in inhibiting the growth of bacteria and a wide variety of viruses and is therefore found a versatile mixture that provides broad spectrum protection to pathogenic infections.

### Example 6—Infant Formula with Mixture of 19 HMOs

Infant formula intended for infants 0-6 month of age, comprising per 100 ml (obtained from reconstituting 13.7 g powder with water):
67 kcal
digestible carbohydrates (mainly lactose): 7.3 g
protein (whey protein, casein): 1.3 g
lipids: 3.4 g
non-digestible oligosaccharides 0.9 g consisting of
   0.7 g GOS/IcFOS in a 7.6:1 wt/wt ratio
   0.2 g of a mixture of 19 HMOs according to example 1 consisting of 28.3 wt % 2′-FL, 21.2 wt % 3-FL, 2.1 wt % 3′-SL, 4.1 wt % 6′-SL, 3.4 wt % DFL, 7.5 wt % LNT, 0.9 wt % LNnT, 3.3 wt % LNFP I, 4.1 wt % LNFPII, 3.3 wt % LNFP III, 0.3 wt % LNFP V, 1.2 wt % LSTa, 1 wt % LSTb, 4.5 wt5 LSTc, 4.6 wt % LNDH I, 0.001 wt % LNnDFH I, 1 wt % LNDFH II and LNnDFH II, 9.3 wt % DSLNT,
micronutrients according to directive for infant formulas

### Example 7—Infant Formula with Mixture of 19 HMOs

Infant formula intended for infants 0-6 month of age, comprising per 100 ml (obtained from reconstituting 13.7 g powder with water):
67 kcal
digestible carbohydrates (mainly lactose): 7.3 g
protein (whey protein, casein): 1.3 g
lipids: 3.4 g
non-digestible oligosaccharides 0.9 g consisting of
   0.32 g GOS/IcFOS in a 2.6:1 wt/wt ratio
   0.58 g of a mixture of 19 HMOs according to example 1 consisting of 28.3 wt % 2′-FL, 21.2 wt % 3-FL, 2.1 wt % 3′-SL, 4.1 wt % 6′-SL, 3.4 wt % DFL, 7.5 wt % LNT, 0.9 wt % LNnT, 3.3 wt % LNFP I, 4.1 wt % LNFPII, 3.3 wt % LNFP III, 0.3 wt % LNFP V, 1.2 wt % LSTa, 1 wt % LSTb, 4.5 wt5 LSTc, 4.6 wt % LNDH I, 0.001 wt % LNnDFH I, 1 wt % LNDFH II and LNnDFH II, 9.3 wt % DSLNT,
micronutrients according to directive for infant formulas

### Example 8: Infant Formula with Mixture of 19 HMOs

Infant formula intended for infants 0-6 month of age, comprising per 100 ml (obtained from reconstituting 13.7 g powder with water):
66 kcal
digestible carbohydrates (mainly lactose): 7.2 g
protein (whey protein, casein): 1.3 g
lipids: 3.4 g
non-digestible oligosaccharides 0.872 g consisting of
   0.7 g GOS/IcFOS in a 9:1 wt/wt ratio
   0.2 g of a combination of 19 HMOs consisting of 28.3 wt % 2′-FL, 21.2 wt % 3-FL, 2.1 wt % 3′-SL, 4.1 wt % 6′-SL, 3.4 wt % DFL, 7.5 wt % LNT, 0.9 wt % LNnT, 3.3 wt % LNFP I, 4.1 wt % LNFPII, 3.3 wt % LNFP III, 0.3 wt % LNFP V, 1.2 wt % LSTa, 1 wt % LSTb, 4.5 wt5 LSTc, 4.6 wt % LNDH I, 0.001 wt % LNnDFH I, 1 wt % LNDFH II and LNnDFH II, 9.3 wt % DSLNT
micronutrients according to directive for infant formulas.

### Example 9: Follow-on Formula with Mixture of 19 HMOs

Follow-on formula intended for infants 6-12 month of age, comprising per 100 ml (obtained from reconstituting 14.4 g powder with water):
68 kcal
digestible carbohydrates (mainly lactose): 8.2 g
protein (whey protein, casein): 1.4 g
lipids: 3.2 g
non-digestible oligosaccharides 0.786 g consisting of
   0.7 g GOS/IcFOS 9:1 wt/wt ratio
   85 mg of a combination of 19 HMOs consisting of 28.3 wt % 2′-FL, 21.2 wt % 3-FL, 2.1 wt % 3′-SL, 4.1 wt % 6′-SL, 3.4 wt % DFL, 7.5 wt % LNT, 0.9 wt % LNnT, 3.3 wt % LNFP I, 4.1 wt % LNFPII, 3.3 wt % LNFP III, 0.3 wt % LNFP V, 1.2 wt % LSTa, 1 wt % LSTb, 4.5 wt5 LSTc, 4.6 wt % LNDH I, 0.001 wt % LNnDFH I, 1 wt % LNDFH II and LNnDFH II, 9.3 wt % DSLNT
micronutrients according to directive for follow-on formulas

### Example 10: Young Child Formula with Mixture of 19 HMOs

Young child formula intended for young children 12 to 47 months of age, comprising per 100 ml (after reconstituting 14.4 g powder with water):
65 kcal
digestible carbohydrates (mainly lactose): 8.3 g
protein (whey protein, casein): 1.3 g
lipids: 2.7 g
non-digestible oligosaccharides: 1.243 g consisting of
   1.2 g GOS/IcFOS 9:1 wt/wt ratio
   43 mg of a combination of 19 HMOs consisting of 28.3 wt % 2′-FL, 21.2 wt % 3-FL, 2.1 wt % 3′-SL, 4.1 wt % 6′-SL, 3.4 wt % DFL, 7.5 wt % LNT, 0.9 wt % LNnT, 3.3 wt % LNFP I, 4.1 wt % LNFPII, 3.3 wt % LNFP III, 0.3 wt % LNFP V, 1.2 wt % LSTa, 1 wt % LSTb, 4.5 wt5 LSTc, 4.6 wt % LNDH I, 0.001 wt % LNnDFH I, 1 wt % LNDFH II and LNnDFH II, 9.3 wt % DSLNT
micronutrients according to directive for follow-on formula.

### Example 11—Effect of HMO Mixture on Clostridium difficile Toxin

To assess the potential of the 19 HMO mixture to provide anti-infective effects to an infection with the gram-positive bacterium Clostridium difficile the effect of HMOs on the gut barrier was assessed using a 3-dimensional gut tubule model.

### Tubular Model of Caco-2 Cells (Organoplate®)

In the method of the present invention, Organoplates were utilized as a three-dimensional model of tubules (organoplate 3 lane 40, Mimetas, Leiden, The Netherlands). The Caco-2 cells were introduced into the chips and maintained in Dulbecco's Modified Eagle Medium (DMEM) culture medium supplemented with high glucose (4.5 g/L), GlutaMAX, and 10% FCS (Gibco). Following a period of three days, a cellular tubule was established. Experimental procedures were initiated on the fourth day post-cellular introduction.

### Human Milk Oligosaccharides

Mixtures of 5 HMOs (2′-FL, 3-FL, LNT, 3′-SL, 6′-SL) and 19 HMOs according to the invention were tested at the same total concentration of 9 g/L.

### Gut Barrier Challenge

To simulate the transition of the intestinal barrier from tight to leaky during C. difficile infection, the tubules were subjected to a challenge with Toxin A (TcdA) derived from Clostridium difficile (SML1154-2UG, Sigma). This toxin was applied at a concentration of 10 ng/mL in media without FCS. This exposure was sustained for a duration of 16 hours, under conditions both with and without the presence of human milk oligosaccharides (HMOs). The Transepithelial Electrical Resistance (TEER) was monitored utilizing the Organoteer® (Mimetas), within the cell incubator. The percentage TEER evolution over time was determined as a quantitative marker for barrier integrity.

### Data Analysis

Each mixture was applied to 5 chips (replicates) across 3 separate experiments. Statistical analysis was performed with SPSS, using an estimated marginal means model to account for variations between experiments. This was followed by a post hoc LSD test.

### Results

The mean TEER of the tubules without intervention was 77.6%, which upon exposure to TcdA significantly decreased (table 4). Exposure of the tubules to TcdA in combination with the 5HMO mixture improved the TEER as compared to the situation wherein the tubules were exposed to the toxin without HMOs. TEER of the tubules exposed to both TcdA and the 19 HMO mixture was even further (and significantly) restored.

TABLE 4
Estimated marginal means of the effect of different HMO
mixtures on TEER evolution.
Estimated % TEER evolution
Dependent Variable: TEER

95% Confidence
Interval

| | | Std. | Lower | Upper |
|---|---|---|---|---|
| Treatment | Mean | Error | Bound | Bound |
| Medium | 77.57% | 3.25% | 71.03% | 84.11% |
| Medium + TcdA | 36.15% | 3.25% | 29.61% | 42.69% |
| HMO5 + TcdA | 43.42% | 3.25% | 36.88% | 49.97% |
| HMO19 + TcdA | 61.66% | 3.39% | 54.85% | 68.47% |

The results show that exposure to the 19 HMO mixture has a preventive or protective effect on C. difficile toxin-induced gut barrier disruption.

### Example 12—Effect of HMO Mixture on a Synthetic dsRNA Virus Proxy

To assess the effect of a 19 HMO mixture on the inflammatory response to infection with a double-stranded RNA virus an in vitro model was employed wherein HT-29 cells were pre-incubated with test compounds and subsequently challenged with a Lyovec-complexed Poly 1:C as a synthetic proxy of a dsRNA infection.

### Materials and Methods

Cell culture: HT-29 cell line (HTB-38™, ATCC) was seeded (65.000 cell/mL; 100 μl per well) in flat bottom 96 well plates (Falcon) using Mc Coy 5A medium (Thermo Fisher scientific, 26600080) supplemented with 10% heat-inactivated FCS (Gibco, A5256701), 1% penicillin/streptomycin (Thermo Fisher Scientific, 15140130). Cells were kept in incubation at 37° C. and 5% CO₂ and medium was refreshed on day 3.

Pre-incubation with test compounds: On day 7, when cells reached confluence, cells were washed (1×) with PBS (Thermo fisher Scientific, 14190144). Then, cells were exposed to either medium (Mc Coy 5A supplemented with 2% heat-inactivated FCS and 1% penicillin/streptomycin) or 1% (w/v) solutions of 2′FL, 5HMOS or 19HMOS for 24 hours.

Stimulation with Poly I:C: After 24 h incubation, medium was removed, and cells were exposed to either fresh medium (Mc Coy 5A supplemented with 2% heat-inactivated FCS and 1% penicillin/streptomycin) or 2 μg/mL Lyovec-complexed Poly I:C (Invivogen, tlrl-picwlv) for additional 24 hours.

Determination of cell viability and IL-8 secretion: After the incubation, supernatants were collected and stored in −20° C. for further analysis. In addition, the viability of the cells was measured by LDH-Glo™ (Promega, J2381) and WST-1 (Merck, 11644807001) following manufacturer's protocol. Results showed no viability issues upon stimulations applied. Human IL-8/CXCL8 DuoSet ELISA kit (R&D systems, DY208) was used to measure IL-8 secretion on conditioned supernatants. Therefore, supernatants were diluted (25×) in reagent diluent and manufacturer's protocol was followed.

### Results

The IL-8 secretion response to a challenge with Poly I:C, a proxy for a dsRNA virus infection, was assessed (table 5). Exposure to Poly I:C resulted in a strong increase in mean IL-8 secretion, which was significantly decreased upon exposure to the mixture of 19 HMOs, an effect that was not observed with 2′-FL or 5 HMOs. The results are indicative of a beneficial prevention of the inflammatory response triggered by viral infection.

TABLE 5
Mean IL-8 secretion in pg/ml of cells exposed to control conditions and cells challenged with
Poly I:C and exposed to cellobiose, lactoferrin, a 5 HMO mixture or a 19 HMO mixture.

| | | | | | 0.1% | 0.1% | | | 1% |
|---|---|---|---|---|---|---|---|---|---|
| | | 0.1% | 0.1% | | Cellobiose + | Lactoferrin + | 1% 2′FL + | 1% 5HMOS + | 19HMOS + |
| | Medium | Cellobiose | Lactoferrin | Poly I:C | Poly I:C | Poly I:C | Poly I:C | Poly I:C | Poly I:C |
| Mean | 359.54 | 277.94 | 343.19 | 21799.28 | 20023.80 | 9201.69 | 33126.64 | 26684.96 | 8276.76 |
| stdev | 60.1 | 54.7 | 5969.8 | 10903.8 | 1214.4 | 12870.7 | 10900.9 | 6174.0 | 10692.4 |

### Example 13—Effect of 19 HMO Mixture on Growth Inhibition of Pathogenic Bacteria

The effect of the total HMO mixture, the mixture of 19 HMOs, a mixture of 5 HMOs and 2′-FL in a concentration between 1 mg/ml to 16 mg/ml to inhibit growth of different bacterial strains (E. coli, S. typhimurium, P. mirabilis and S. aureus) was assessed by growth percentage determination and K-factor inhibition was determined.

Bacteria were inoculated in 5 ml of YCFA broth supplemented with glucose as the sole energy source (YCFAG) and incubated anaerobically at 37° C. for 24 h. Subsequently, the strain was propagated to 5 ml of YCFAG broth culture overnight as pre-culture; after that, different strains were added to the tubes of YCFA broth supplemented with the different carbon sources at a ratio of 1:50 in duplicate at 37° C. and at pH 5.5. Growth in cultures was monitored spectrophotometrically from 0 to 24-h culture by measuring the OD600 by using Ultrospec 10 cell density meter (Amersham Biosciences GmbH, Germany).

### Calculation of Area Under the Curve (AUC)

The Area Under the Curve (AUC) was calculated using samples collected up to the timepoint corresponding to the reference line. The reference line was determined as the average of (N=10) measurements taken during the early stationary phase of the growth curve. All calculations were performed using GraphPad Prism software. A higher AUC is thus indicative for less or no growth inhibition and vice versa.

Growth Factor (k-factor) Calculation

The growth factor, denoted as the (k)-factor, was determined using a non-linear fit to a logistic growth model. The k-factor indicates the growth rate of the bacterial population. This analysis was also conducted using GraphPad Prism software. The logistic growth model used is defined by the equation: Y=(YM-Y0)·exp(−k·x)+Y0YM·Y0. A used therein (Y0) is the starting population, (YM) is the maximum population, (k) is the rate constant (inverse units of (X)), (frac{1}{k}) is the (X)-coordinate of the first inflection point, representing the time at which the growth rate begins to decelerate.

E coli E374

The effect of the HMO mixtures on growth inhibition of E. coli E374 (ETEC, ATCC 35401) were assessed and on the basis of the growth curves the AUC and k-factor were calculated (tables 6 and 7 respectively).

TABLE 6
Percentage growth inhibition based on AUC of E. coli 374 exposed to total
HMOs, 19 HMOs, 5 HMOs and 2′-FL at different concentrations.
E374% inhibition AUC

| mg/ml | Tot HMO | HMO19 | HMO5 | 2FL |
|---|---|---|---|---|
| 16 | 42 | 55 | 11 | 13 |
| 8 | 20 | 24 | 11 | 15 |
| 4 | 3 | 12 | 8 | 11 |
| 2 | 4 | 6 | 10 | 12 |
| 1 | −8 | −5 | −3 | 2 |
| 0 | 0 | 0 | 0 | 0 |

TABLE 7
Percentage growth inhibition expressed as the K-factor of E. coli 374
exposed to total HMOs, 19 HMOs, 5 HMOs and 2′-FL
at different concentrations.
E374% inhibition K-factor

| mg/ml | Tot HMO | HMO19 | HMO5 | 2FL |
|---|---|---|---|---|
| 16 | 46 | 76 | 14 | 16 |
| 8 | 22 | 48 | 12 | 13 |
| 4 | 11 | 25 | 8 | 7 |
| 2 | 8 | 19 | 11 | 11 |
| 1 | 0 | 5 | −1 | 3 |
| 0 | 0 | 0 | 0 | 0 |

A dose dependent inhibition of the growth of E. coli was observed with the mixture of 19 HMOs from a low dose onwards, both the percentage inhibition and the inhibition K-factor of 19 HMOs were higher than the inhibition observed for the total HMOs. The results are indicative of the anti-infective potential of the mixture of 19 HMOs according to the invention.

### S. Aureus S17

The effect of the HMO mixtures on growth inhibition of S. aureus S17 (ATCC 29213) were assessed and on the basis of the growth curves the AUC, percentage growth inhibition and k-factor were calculated (tables 8 and 9 respectively).

TABLE 8
Percentage growth inhibition based on AUC of S. aureus S17 exposed to
total HMOs, 19 HMOs, 5 HMOs and 2′-FL at different concentrations.
S17% inhibition AUC

| mg/ml | Tot HMO | HMO19 | HMO5 | 2FL |
|---|---|---|---|---|
| 16 | 53 | 45 | 30 | 37 |
| 8 | 41 | 38 | 35 | 31 |
| 4 | 31 | 21 | 33 | 36 |
| 2 | 29 | 14 | 25 | 29 |
| 1 | 7 | 100 | 15 | 19 |
| 0 | 0 | 0 | 0 | 0 |

TABLE 9
Percentage growth inhibition expressed as the K-factor of S. aureus S17
exposed to total HMOs, 19 HMOs, 5 HMOs and 2′-FL
at different concentrations.
S17% inhibition K-factor

| mg/ml | Tot HMO | HMO19 | HMO5 | 2FL |
|---|---|---|---|---|
| 16 | 49 | 49 | 26 | 27 |
| 8 | 39 | 35 | 27 | 27 |
| 4 | 23 | 17 | 20 | 23 |
| 2 | −1 | 19 | 12 | 18 |
| 1 | 9 | 100 | 12 | 16 |
| 0 | 0 | 0 | 0 | 0 |

Inhibition of the growth of S. aureus S17 was observed with both the mixture of 19 HMOs and total HMOs in a dose dependent manner showing significant inhibition at 8 mg/ml and 16 mg/ml. The percentage inhibition and the inhibition K-factor of 19 HMOs are similar to the inhibition observed for the total HMOs. The results support the broad anti-infective potential of the mixture of 19 HMOs according to the invention.

S. typhimurium S19

The effect of the HMO mixtures on growth inhibition of S. typhimurium S19 (ATCC 14028) were assessed and on the basis of the growth curves the AUC, percentage growth inhibition and k-factor were calculated (tables 10 and 11 respectively).

TABLE 10
Percentage growth inhibition based on AUC of S. typhimurium S19
exposed to total HMOs, 19 HMOs, 5 HMOs and 2′-FL
at different concentrations.
S19% inhibition AUC

| mg/ml | Tot HMO | HMO19 | HMO5 | 2FL |
|---|---|---|---|---|
| 16 | 37 | 50 | 18 | 18 |
| 8 | 24 | 31 | 14 | 15 |
| 4 | 12 | 17 | 14 | 13 |
| 2 | 6 | 10 | 9 | 14 |
| 1 | 3 | 5 | 5 | 7 |
| 0 | 0 | 0 | 0 | 0 |

TABLE 11
Percentage growth inhibition expressed as the K-factor of S. typhimurium
S19 exposed to total HMOs, 19 HMOs, 5 HMOs and 2′-FL
at different concentrations.
S19% inhibition K-factor

| mg/ml | Tot HMO | HMO19 | HMO5 | 2FL |
|---|---|---|---|---|
| 16 | 52 | 71 | 25 | 26 |
| 8 | 38 | 60 | 23 | 25 |
| 4 | 21 | 32 | 19 | 23 |
| 2 | 9 | 21 | 13 | 19 |
| 1 | 9 | 15 | 9 | 10 |
| 0 | 0 | 0 | 0 | 0 |

A dose dependent inhibition of the growth of S. typhimurium S19 was observed with the mixture of 19 HMOs from a low dose onwards, wherein both the percentage inhibition and the inhibition K-factor of 19 HMOs were higher than the inhibition observed for the total HMOs. The results are indicative of the broad and strong anti-infective potential of the mixture of 19 HMOs according to the invention.

P. mirabilis P80

The effect of the HMO mixtures on growth inhibition of P. mirabilis P80 were assessed and on the basis of the growth curves the AUC, percentage growth inhibition and k-factor were calculated (tables 12 and 13 respectively).

TABLE 12
Percentage growth inhibition based on AUC of P. mirabilis P80
exposed to total HMOs, 19 HMOs, 5 HMOs and 2′-FL
at different concentrations.
P80% inhibition AUC

| mg/ml | Tot HMO | HMO18 | HMO5 | 2FL |
|---|---|---|---|---|
| 16 | 48 | 40 | 17 | 21 |
| 8 | 27 | 16 | 6 | 10 |
| 4 | 4 | 7 | 10 | 7 |
| 2 | 5 | 6 | 3 | 7 |
| 1 | 0 | 0 | 3 | 0 |
| 0 | 0 | 0 | 0 | 0 |

TABLE 13
Percentage growth inhibition expressed as the K-factor of S. typhimurium
S19 exposed to total HMOs, 19 HMOs, 5 HMOs and 2′-FL
at different concentrations.
P80% inhibition K-factor

| mg/ml | Tot HMO | HMO18 | HMO5 | 2FL |
|---|---|---|---|---|
| 16 | 59 | 61 | 22 | 30 |
| 8 | 42 | 39 | 27 | 26 |
| 4 | 31 | 28 | 25 | 21 |
| 2 | 4 | 14 | 6 | 15 |
| 1 | 1 | 6 | −1 | −4 |
| 0 | 0 | 0 | 0 | 0 |

Inhibition of the growth of P. mirabilis P80 was observed with both the mixture of 19 HMOs and total HMOs in a dose dependent manner showing inhibition from 2 mg/ml onwards. The percentage inhibition and the inhibition K-factor of 19 HMOs are similar to the inhibition observed for the total HMOs.

The results support the broad anti-infective potential of the mixture of 19 HMOs according to the invention wherein the 19 HMO mixture shows broad anti-infection potential against both gram-negative pathogenic bacteria E. coli, S. typhimurium and P. mirabilis, as well as gram-positive bacteria S. aureus.

1. Synthetic mixture of HMOs wherein the mixture comprises 10-30 wt % acidic HMOs and 70-90 wt % neutral HMOs, wherein the neutral HMOs comprises 75-95 wt % of fucosylated HMOs based on weight of the neutral HMOs and wherein the mixture comprises at least 19 HMOs.

2. Synthetic mixture of HMOs according to claim 1 wherein the acidic HMOs comprise at least 3′-sialyllactose (3′-SL) and 6′-sialyllactose (6′-SL) and wherein the weight ratio of 3′SL to 6′SL is between 0.5:3 to 1:1, preferably between 1:3 to 1:2, even more preferably about 1:2.

3. Synthetic mixture of HMOs according to any one of claims 1 and 2 comprising 3′SL, 6′SL, LSTa, LSTb, LSTc, DSLNT, 2′FL, 3′FL, DFL, LNT, LNnT, LNFP I, LNFP II, LNFP III, LNFP V, LNDFH I, LNnDFH I and LNDFH II and LNnDFH II.

4. Synthetic mixture of HMOs according to any one of claim 1 to 3 wherein of HMOs comprises, preferably consists essentially of
(i) 1.4 to 2.6 wt % 3′-SL, more preferably 1.6 to 2.4 wt %, even more preferably 1.8 to 2.2 wt %;
(ii) 3.5 to 4.7 wt % 6′SL, more preferably 3.7 to 4.5 wt %, even more preferably 3.9 to 4.3 wt %;
(iii) 0.8 to 1.6 wt % LSTa, more preferably 0.9 to 1.5 wt %, even more preferably 1.0 to 1.4 wt %;
(iv) 0.7 to 1.4 wt % LSTb, more preferably 0.8 to 1.3 wt %, even more preferably 0.9 to 1.2 wt %;
(v) 4.1 to 4.8 wt % LSTc, more preferably 4.2 to 4.7 wt %, even more preferably 4.3 to 4.6 wt %;
(vi) 7.8 to 11.2 wt % DSLNT, more preferably 8.0 to 11.0 wt %, even more preferably 8.5 to 10.5 wt %;
(vii) 25 to 32 wt % 2′FL, more preferably 26 to 31 wt %, even more preferably 27 to 30 wt %;
(viii) 19 to 25 wt % 3′FL, more preferably 20 to 24 wt %, even more preferably 20.5 to 23 wt %;
(ix) 4.1 to 4.8 wt % DFL, more preferably 4.2 to 4.7 wt %, even more preferably 4.3 to 4.6 wt %;
(x) 5 to 10 wt % LNT, more preferably 6 to 9 wt %, even more preferably 7 to 8 wt %;
(xi) 0.5 to 1.1 wt % LNnT, more preferably 0.6 to 1 wt %, even more preferably 0.7 to 0.9 wt %;
(xii) 2.9 to 3.6 wt % LNFP I, more preferably 3 to 3.5 wt %, even more preferably 3.1 to 3.4 wt %;
(xiii) 3.8 to 4.4 wt % LNFP II, more preferably 3.9 to 4.3 wt %, even more preferably 4 to 4.2 wt % (xiv) 2.9 to 3.6 wt % LNFP III, more preferably 3 to 3.5 wt %, even more preferably 3.1 to 3.4 wt %;
(xv) 0.2 to 0.5 wt % LNFP V, more preferably 0.25 to 0.45 wt %, even more preferably 0.3 to 0.4 wt %;
(xvi) 4.2 to 5 wt % LNDFH I, more preferably 4.4 to 4.8 wt %, even more preferably 4.5 to 4.75 wt %;
(xvii) 0.00001 to 0.02 wt % LNnDFH I, more preferably 0.0001 to 0.015 wt %, even more preferably 0.001 to 0.01 wt %; and
(xviii) 0.7 to 1.3 wt of the combination of LNDFH II and LNnDFH II, more preferably 0.8 to 1.2 wt %, even more preferably 0.9 to 1.15 wt %;
based on total dry weight of the synthetic HMO mixture.

5. Nutritional composition comprising the synthetic HMO mixture according to any one of claim 1 to 4, wherein the nutritional composition is selected from infant formula, follow-on formula, and young child milk.

6. A synthetic mixture of human milk oligosaccharides (HMOs) comprising 10-30 wt % acidic HMOs and 70-90 wt % neutral HMOs wherein the neutral HMOs comprises 75-95 wt % of fucosylated HMOs based on weight of the neutral HMOs for use in the treatment and/or prevention of infections, preferably pathogenic infections, wherein the mixture preferably comprises at least 19 HMOs.

7. The synthetic mixture of HMOs for use according to claim 6, wherein the acidic HMOs comprise at least 3′-sialyllactose (3′-SL) and 6′-sialyllactose (6′-SL) and wherein the weight ratio of 3′SL to 6′SL is between 0.5:3 to 1:1, preferably between 1:3 to 1:2, even more preferably about 1:2.

8. The synthetic mixture of HMOs for use according to claims 6 and 7, wherein the use in the treatment and/or prevention of infections, preferably pathogenic infections with a virus selected from double-stranded RNA viruses, single-stranded positive and negative sense RNA viruses, and DNA viruses and/or bacteria selected from gram-positive and gram-negative bacteria.

9. The synthetic mixture of HMOs for use according to any one of claims 6 to 8, wherein the use in the treatment and/or prevention of infections, preferably pathogenic infections is against a virus selected from rotavirus, respiratory syncytial virus (RSV) and SARS-COV2 and/or bacterium selected from L. monocytogenes, E. coli, S. aureus, S. typhimurium, L. monocytogenes, E. faecium, E. faecalis, P. mirabilis and K. pneumoniae, preferably E. coli.

10. The synthetic mixture of HMOs for use according to any one of the claims 6 to 9 wherein the treatment and/or prevention of infections, preferably pathogenic infections is more similar to the treatment and/or prevention of infections observed in human milk fed infants and/or improved compared to the treatment and/or prevention of infections in infants administered a composition not comprising the mixture of HMOs.

11. The synthetic mixture of HMOs for use according to any one of claims 6 to 10, wherein the neutral HMOs comprise at least LNT and LNnT and wherein the weight ratio of LNT to LNnT is between 12:1 and 1:1, preferably between 10:1 and 3:1, even more preferably between 9:1 to 6:1.

12. The synthetic mixture of HMOs for use according to any one of claims 6 to 11, wherein HMOs are selected from 3′SL, 6′SL, LSTa, LSTb, LSTc, DSLNT, 2′FL, 3′FL, DFL, LNT, LNnT, LNFP I, LNFP II, LNFP III, LNFP V, LNDFH I, LNnDFH I, LNDFH II and LNnDFH II.

13. The synthetic mixture of HMOs for use according to any one claims 6 to 12, wherein the mixture of HMOs is comprised in a nutritional composition, wherein the nutritional composition is selected from infant formula, follow-on formula, and young-child formula.

14. Nutritional composition comprising the synthetic mixture of HMOs for use according to any one of the claims 6 to 13, wherein the nutritional composition further comprising group II non-digestible saccharides selected from the group consisting of fructo-oligosaccharides, non-digestible dextrins, galacto-oligosaccharides (such as betagalacto-oligosaccharides), xylo-oligosaccharides, arabino-oligosaccharides, arabinogalacto-oligosaccharides, gluco-oligosaccharides, gentio-oligosaccharides, glucomanno-oligosaccharides, galactomanno-oligosaccharides, mannan-oligosaccharides, isomalto-oligosaccharides, nigero-oligosaccharides, chito-oligosaccharides, soy oligosaccharides, uronic acid oligosaccharides, and mixtures thereof, and wherein the total weight ratio of the mixture of HMOs and the group II non-digestible saccharides of short chain oligosaccharides having a DP between 3 and 6 (DP 3-6) to long chain oligosaccharides have a DP of 7 and higher is from 5:1 to 12:1, preferably from 8:1 to 10:1, even more preferably in a ratio of about 9:1.

15. The synthetic mixture of HMOs for use according to claims 6 to 13 and the nutritional composition for use according to claim 14, wherein the use comprises improving gut barrier integrity and/or preventing gut barrier disruption.
